(19) United States
(12) Patent Application Publication
(10) Pub. No.: US 2026/0242473 A1
(43) Pub. Date: Aug. 20, 2026

(54) METHODS FOR REDUCING EXOGENOUS INSULIN USE

(71) Applicant: Provention Bio, Inc., Red Bank, NJ (US)

(72) Inventors: Eleanor L. Ramos, Pacifica, CA (US); Ralph Raymond, Red Bank, NJ (US)

(73) Assignee: Provention Bio, Inc., Red Bank, NJ (US)

(21) Appl. No.: 19/471,172

(22) PCT Filed: Mar. 29, 2024
(86) PCT No.: PCT/US2024/022121

§ 371 Date: Sep. 30, 2025

Related U.S. Application Data
(60) Provisional application No. 63/493,113, filed on Mar. 30, 2023.

(51) Int. Cl. C07K 16/28; A61K 39/00; A61P 3/10

(52) U.S. Cl. CPC C07K 16/2809; A61P 3/10; A61K 2039/505; A61K 2039/54; A61K 2039/545; C07K 2317/24; C07K 2317/52; C07K 2317/565

(57) ABSTRACT

Provided herein are a method of reducing exogenous insulin use. In some embodiments, such method can include administering to a subject in need thereof a 12-day to 14-day course of teplizumab at a total dose of from about 9000 μg/m² and about 14000 μg/m².

### CROSS-REFERENCE TO RELATED APPLICATION

This application claims priority from U.S. Provisional Application 63/493,113, filed Mar. 30, 2023, the content of which is incorporated herein by reference in its entirety.

### SEQUENCE LISTING

The application contains a Sequence Listing which has been submitted electronically in .XML format and is hereby incorporated by reference in its entirety. Said. XML copy, created on Mar. 22, 2024, is named “122548. WO060.xml” and is 3,387 bytes in size.

### BACKGROUND

Type 1 diabetes (T1D) is caused by the autoimmune destruction of insulin-producing beta cells in the islets of Langerhans, leading to dependence on exogeneous insulin injections for survival. Approximately 1.6 million Americans have T1D, and after asthma, it remains one of the most common diseases of childhood. Despite improvements in care, most affected individuals with T1D are not able to consistently achieve desired glycemic targets. For individuals with T1D, there are persisting concerns for increased risk of both morbidity and mortality.

### SUMMARY

Some aspects of the present disclosure relate to a method of reducing exogenous insulin use in a subject in need thereof, the method comprising administering to the subject a 12-day to 14-day course of teplizumab at a total dose of from about 9000 μg/m² and about 14000 μg/m², wherein administration of teplizumab results in a reduction of exogenous insulin use by at least 0.08 U/kg/day.

In some embodiments, the administration of teplizumab results in a reduction of exogenous insulin use by at least 0.08 U/kg/day at year 1. In some embodiments, the administration of teplizumab results in a reduction of exogenous insulin use by at least 0.10 U/kg/day at year 2.

In some embodiments, the method comprises administering to the course of teplizumab. In further embodiments, the 12-day course of teplizumab comprises a first dose of about 106 μg/m² teplizumab on day 1, a second dose of about 425 μg/m² teplizumab on day 2, and one dose of about 850 μg/m² teplizumab on each of days 3-12.

In other embodiments, the method comprises administering to the subject a 14-day course of teplizumab. In further embodiments, the 14-day course of teplizumab comprises a first dose of about 100 μg/m² teplizumab on day 1, a second dose of about 425 μg/m² teplizumab on day 2, a third dose of about 850 μg/m² teplizumab on day 3, a four dose of about 850 μg/m² teplizumab on day 4, and a dose of about 1000 μg/m² teplizumab on each of days 5-14.

In some embodiments, the method comprises administering a first and a second 12-day to 14-day courses of teplizumab. In some embodiments, the first and the second 12-day to 14-day courses are administered at about 6 months interval.

In some embodiments, the method comprises administering a first and a second 12-day courses of teplizumab. In some embodiments, the first and the second 12-day courses are administered at about 6 months interval.

In some embodiments, the subject in need thereof is about 7.5 years old or older.

In some embodiments, the method comprises administering teplizumab by intravenous infusion.

In some embodiments, reduction of exogenous insulin use is over a period of 1 year or more.

Some aspects of the present disclosure relate to teplizumab for use in a method of reducing exogenous insulin use in a subject in need thereof, the method comprising administering to the subject a 12-day to 14-day course of teplizumab at a total dose of from about 9000 μg/m² and about 14000 μg/m², wherein administration of teplizumab results in a reduction of exogenous insulin use by at least 0.08 U/kg/day.

Also provided in the present disclosure is use of teplizumab for the manufacture of a medicament for reducing exogenous insulin use in a subject in need thereof in a method herein.

### BRIEF DESCRIPTION OF THE DRAWINGS

FIG. 1: Forest plot of least-squares mean differences between teplizumab and control group changes in the change from baseline C-peptide AUC. Panel A: The analysis includes the 5 clinical trials with year 1 data. The analysis is with and without the imputed data for missing values. Panel B: The analysis includes the 3 clinical trials and 2 data were available. The analysis is with and without the imputed data for missing values.

FIG. 2: Mean (SE) Observed C-peptide and insulin use from the clinical trials. Panel A: C-peptide AUC levels at 1 year from the 5 studies with 1-year data. The p-value at 1 year was derived from the ANCOVA model for the 1 year integrated observed data. Panel B: C-peptide AUC levels at 2 years from the 3 studies with year 2 data. The p-value at 2 years was derived from the ANCOVA for the 2-year integrated observed data. Panel C: Average Insulin Use at 1 year. The p-value at 1 year was derived from the ANCOVA for the 1 year integrated observed data. Panel D: Average Insulin Use at 2 years. The p-value at 2 years was derived from the ANCOVA for the 2-year integrated observed data.

FIG. 3: Forest Plot of Least-Squares Mean Differences Between Teplizumab and Control groups in the change from baseline in insulin use. Panel A: The analysis includes the 5 clinical trials with year 1 data. The analysis is with and without the imputed data for missing values. Panel B: The analysis includes the 3 clinical trials for which year 1 and 2 data were available. The analysis is with and without the imputed data for missing values.

### DETAILED DESCRIPTION

### I. Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

As used herein, the term “onset” of disease with reference to Type-1 diabetes refers to a patient meeting the criteria established for diagnosis of Type-1 diabetes by the American Diabetes Association (see, Mayfield et al., 2006, Am. Fam. Physician 58:1355-1362).

As used herein, a “protocol” includes dosing schedules and dosing regimens. The protocols herein are methods of use and include therapeutic protocols. A “dosing regimen”, “dosage regimen” or “course of treatment” may include administration of several doses of a therapeutic agent over 1 to 20 days.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human. In some embodiments, the patient population comprises children. In some embodiments, the patient population comprises children newly diagnosed with T1D. In some embodiments, the patient population is treated within 6 weeks of the T1D diagnosis. In some embodiments, the patient population comprises children who are positive for at least one T1D-associated autoantibody and have a peak stimulated C-peptide of ≥0.2 pmol/mL at screening.

As used herein, the term “children” (and variations thereof) includes those being around 8 to 17 years of age.

As used herein, the term “effective amount” refers to that amount of teplizumab sufficient to result in the delay or prevention of the development, recurrence or more symptoms of T1D.

As used herein, the terms “treat”, “treatment” and “treating” refer to the amelioration of one or more symptoms associated with T1D that results from the administration of one or more CD3 binding molecules. In some embodiments, such terms refer to a reduction in a human's average number of hypoglycemic episodes. In other embodiments, such terms refer to the maintenance of a reference level of C-peptide in the peripheral blood.

In some embodiments, the effective amount reduces one or more T1D symptoms by at least 5%, by at least 10%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%.

Various aspects of the disclosure are described in further detail below. Additional definitions are set out throughout the specification.

### II. Anti-CD3 Antibodies and Pharmaceutical Compositions

The terms “anti-CD3 antibody” and “an antibody that binds to CD3” refer to an antibody or antibody fragment that is capable of binding cluster of differentiation 3 (CD3) with sufficient affinity such that the antibody is useful as a prophylactic, diagnostic and/or therapeutic agent in targeting CD3. In some embodiments, the extent of binding of an anti-CD3 antibody to an unrelated, non-CD3 protein is less than about 10% of the binding of the antibody to CD3 as measured, e.g., by a radioimmunoassay (RIA). In some embodiments, an antibody that binds to CD3 has a dissociation constant (Kd) of <1 μM, <100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M). In some embodiments, an anti-CD3 antibody binds to an epitope of CD3 that is conserved among CD3 from different species.

In some embodiments, the anti-CD3 antibody can be ChAglyCD3 (otelixizumab). Otelixizumab is a humanized Fc nonbinding anti-CD3, which was evaluated initially in phase 2 studies by the Belgian Diabetes Registry (BDR) and then developed by Tolerx, which then partnered with GSK to conduct the phase 3 DEFEND new onset T1D trials (NCT00678886, NCT01123083, NCT00763451). Otelixizumab is administered IV with infusions over 8 days. See, e.g., Wiczling et al., J. Clin. Pharmacol. (2010) 50(5):494-506; Keymeulen et al., N Engl J Med. (2005) 352:2598-608; Keymeulen et al., Diabetologia. (2010) 53:614-23; Hagopian et al., Diabetes. (2013) 62:3901-8; Aronson et al., Diabetes Care 54; Ambery et al., Diabet Med. (2014) 31:399-402; Bolt et al., Eur. J. Immunol. (1993) 23:403-11; Vlasakakis et al., Br J Clin Pharmacol (2019) 85:704-14; Guglielmi et al, Expert Opin Biol Ther. (2016) 16(6):841-6; Keymeulen et al., N Engl J Med. (2005) 352:2598-608; Keymeulen et al., Blood (2010) 115(6):1145-55; Sprangers et al., Immunotherapy (2011) 3(11):1303-6; and Daifotis et al., Clinical Immunology (2013) 149:268-78.

In some embodiments, the anti-CD3 antibody can be visilizumab (also called HuM291; Nuvion). Visilizumab is a humanized anti-CD3 monoclonal antibody characterized by a mutated IgG2 isotype, lack of binding to Fcγ receptors, and the ability to induce apoptosis selectively in activated T cells. It was evaluated in patients in graft-versus-host disease (NCT00720629; NCT00032279) and in ulcerative colitis (NCT00267306) and Crohn's Disease (NCT00267709). See, e.g., Sandborn et al., (2010) Gut 59(11):1485-92.

### III. Teplizumab

In some embodiments, the anti-CD3 antibody can be teplizumab. Teplizumab, also known as hOKT3yl (Ala-Ala) (containing an alanine at positions 234 and 235) is an anti-CD3 antibody that had been engineered to alter the function of the T lymphocytes that mediate the destruction of the insulin-producing beta cells of the islets of the pancreas. Teplizumab binds to an epitope of the CD38 chain expressed on mature T cells and by doing so changes their function. Circulating T cells (and other lymphocytes) are transiently reduced following teplizumab treatment, in a process that may include margination and depletion (Long 2017, Sherry 2011). In addition to reduced effector function of T cells, teplizumab appears to both increase the number and function of regulatory T cells (Tregs) (Ablamunits 2010, Bisikirska 2005, Long 2017, Waldron-Lynch 2012). More recent studies indicate that teplizumab induces immunologic “exhaustion” in a subset of effector CD8+ T cells, perhaps making them more susceptible to regulation or deletion (Long 2016, Long 2017). Taken together, these mechanistic data suggest that teplizumab not only exerts a “suppressive” effect on β cell immune destructive processes but rather is an immune “modulator” favoring a rebalancing of effector and regulatory arms involved with T1D autoimmunity and supporting the notion that teplizumab may have the ability to contribute to the re-introduction of β cell self-tolerance (Lebastchi 2013).

Sequences and compositions of teplizumab are disclosed in U.S. Pat. Nos. 6,491,916; 8,663,634; and 9,056,906. The molecular weight of teplizumab is approximately 150 KD. The full sequences of light and heavy chains are set forth below. Bolded portions are the complementarity determining regions.

Teplizumab Light Chain (SEQ ID NO: 1):
DIQMTQSPSSLSASVGDRVTITCSASSSVSYMNWYQQTPGKAPKR
WIYDTSKLASGVPSRFSGSGSGTDYTFTISSLQPEDIATYYCQQW
SSNPFTFGQGTKLQITRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
Teplizumab Heavy Chain (SEQ ID NO: 2):
QVQLVQSGGGVVQPGRSLRLSCKASGYTFTRYTMHWVRQAPGKGL
EWIGYINPSRGYTNYNQKVKDRFTISRDNSKNTAFLQMDSLRPED
TGVYFCARYYDDHYCLDYWGQGTPVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPEAAGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVMHEALHNHYTQKSLSLSPGK

In some embodiments, provided herein, is a pharmaceutical composition. Such compositions comprise an effective amount of an anti-CD3 antibody, and a pharmaceutically acceptable carrier. In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like (See, for example, Handbook of Pharmaceutical Excipients, Arthur H. Kibbe (ed., 2000, which is incorporated by reference herein in its entirety), Am. Pharmaceutical Association, Washington, D.C.

The composition, if desired, can also contain minor amounts of wetting or emulsifying agents, or pH buffering agents. These compositions can take the form of solutions, suspensions, emulsion, tablets, pills, capsules, powders, sustained formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions contain a therapeutically effective amount of a therapeutic agent preferably in purified form, together with a suitable amount of carrier so as to provide the form for proper administration to the patient. The formulation should suit the mode of administration. In some embodiments, the pharmaceutical compositions are sterile and in suitable form for administration to a subject, preferably an animal subject, more preferably a mammalian subject, and most preferably a human subject.

In some embodiments, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering the anti-CD3 antibody, care must be taken to use materials to which the anti-CD3 antibody does not absorb.

In some embodiments, the composition can be delivered in a vesicle, in particular a liposome (see Langer, Science (1990) 249:1527-33; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In some embodiments, the composition can be delivered in a controlled release or sustained release system. In some embodiments, a pump may be used to achieve controlled or sustained release (see Langer, supra; Sefton, 1987, CRC Crit. Ref. Biomed. Eng. 14:20; Buchwald et al., (1980) Surgery 88:507; Saudek et al., (1989) N. Engl. J. Med. 321:574). In some embodiments, polymeric materials can be used to achieve controlled or sustained release of the antibodies of the disclosure or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J Macromol. Sci. Rev. Macromol. Chem. (1983) 23:61; see also Levy et al., (1985) Science 228:190; During et al., (1989) Ann Neurol. 25:351; Howard et al., (1989) J Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but a poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the disclosure or fragments thereof. See, e.g., U.S. Pat. No. 4,526,938; PCT Publication No. WO 91/05548; PCT Publication No. WO 96/20698; Ning et al., (1996) Radiotherapy & Oncology 39:179-89; Song et al., (1995) PDA Journal of Pharmaceutical Science & Technology 50:372-397; Cleek et al., (1997) Pro Int'l Symp Control Rel Bioact Mater. 24:853-4; and Lam et al., (1997) Proc Int'l Symp Control Rel Bioact Mater. 24:759-760.

A pharmaceutical composition can be formulated to be compatible with its intended route of administration. Examples of routes of administration include, but are not limited to, parenteral, e.g., intravenous, intradermal, subcutaneous, oral, intranasal (e.g., inhalation), transdermal (topical), transmucosal, and rectal administration. In some embodiments, the composition is formulated in accordance with routine procedures as a pharmaceutical composition adapted for intravenous, subcutaneous, intramuscular, oral, intranasal or topical administration to human beings. In some embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder formulation with a suitable vehicle, e.g., sterile pyrogen-free water, before use.

In some embodiments, the disclosure provides dosage forms that permit administration of the anti-CD3 antibody continuously over a period of hours or days (e.g., associated with a pump or other device for such delivery), for example, over a period of 1 hour, 2 hours, 3 hours, 4 hours, 6 hours, 8 hours, 10 hours, 12 hours, 16 hours, 20 hours, 24 hours, 30 hours, 36 hours, 4 days, 5 days, 7 days, 10 days or 12 days. In some embodiments, the disclosure provides dosage forms that permit administration of a continuously increasing dose, for example, increasing from 106 μg/m²/day to 850 μg/m²/day or 211 μg/m²/day to 840 μg/m²/day over a period of 24 hours, 30 hours, 36 hours, 4 days, 5 days, 7 days, 10 days or 12 days.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of the compositions disclosed herein are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the disclosure provides that the anti-CD3 antibodies, or pharmaceutical compositions thereof, can be packaged in a hermetically sealed container such as an ampoule or sachet indicating the quantity of the agent. In some embodiments, the anti-CD3 antibody, or pharmaceutical compositions thereof is supplied as a dry sterilized lyophilized powder or water free concentrate in a hermetically sealed container and can be reconstituted, e.g., with water or saline to the appropriate concentration for administration to a subject. Preferably, the anti-CD3 antibody, or pharmaceutical compositions thereof is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized agents, or pharmaceutical compositions herein should be stored at between its original container and the therapeutic agents, or pharmaceutical compositions of the disclosure should be administered within 1 week, preferably within 5 days, within 72 hours, within 48 hours, within 24 hours, within 12 hours, within 6 hours, within 5 hours, within 3 hours, or within 1 hour after being reconstituted. In some embodiments, the pharmaceutical composition is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

In some embodiments, the disclosure provides that the composition of the disclosure is packaged in a hermetically sealed container such as an ampoule or sachet indicating the quantity of the anti-CD3 antibody.

The compositions may, if desired, be presented in a pack or dispenser device that may contain one or more unit dosage forms containing the active ingredient. The pack may, for example, comprise metal or plastic foil, such as a blister pack.

The amount of the composition of the disclosure which is effective in the treatment of one or more symptoms associated with T1D can be determined by standard clinical techniques. The precise dose to be employed in the formulation can also depend on the route of administration and the seriousness of the condition, and should be decided according to the judgment of the practitioner and each patient's circumstances. Effective doses may be extrapolated from dose-response curves derived from in vitro or animal model test systems.

### IV. Methods and Use

Method of reducing exogenous insulin use in a subject in need thereof are provided herein. In some embodiments, the method comprises administering to the subject a 12-day to 14-day course of teplizumab at a total dose of from about 9000 μg/m² and about 14000 μg/m², wherein administration of teplizumab results in a reduction of exogenous insulin use by at least 0.08 U/kg/day.

In some embodiments, the administration of teplizumab results in a reduction of exogenous insulin use by at least 0.08 U/kg/day at year 1. In some embodiments, the administration of teplizumab results in a reduction of exogenous insulin use by at least 0.10 U/kg/day at year 2.

In some embodiments, the administration of teplizumab results in preservation of C-peptide levels as reflected by greater endogenous insulin production and less exogenous insulin requirement.

In some embodiments, the method comprises administering to the subject a 12-day course of teplizumab. In other embodiments, the method comprises administering to the subject a 14-day course of teplizumab.

In some embodiments, the method comprises administering a first and a second 12-day to 14-day courses of teplizumab. In some embodiments, the first and the second 12-day to 14-day courses are administered at about 6 months interval.

In some embodiments, the method comprises administering a first and a second 12-day courses of teplizumab. In some embodiments, the first and the second 12-day courses are administered at about 6 months interval.

In some embodiments, the subject in need thereof has stage 3 type 1 diabetes. In some embodiments, the subject in need thereof has been diagnosed with stage 3 type 1 diabetes within 5 weeks to 31 weeks prior to the administrating step. In some embodiments, the subject in need thereof is about 7.5 years old or older.

In some embodiments, the method comprises administering teplizumab by intravenous infusion.

In some embodiments, reduction of exogenous insulin use is over a period of 1 year, 2 years or more.

In some embodiments, the method provided herein result in reduced insulin requirements or independence from exogenous insulin.

Type 1 diabetes usually develops in childhood and adolescence; however, it can also present in adulthood as late as the 5th and 6th decades of life, although much less frequently (Atkinson 2014, Bluestone 2010, Streisand 2014).

In some embodiments, anti-human CD3 antibodies such as teplizumab is administered to patients 8 through 17 years old 6 weeks from T1D diagnosis having a peak C-peptide level of ≥0.2 pmol/mL during a mixed meal tolerance test (MMTT). In some embodiments, the peak C-peptide level at screening rages from 0.2 pmol/mL (inclusive) to 0.7 pmol/mL (inclusive).

In some embodiments, T1D diagnosis is according to the American Diabetes Association (ADA) criteria. As defined by the American Diabetes Association (ADA) for the clinical diagnosis of diabetes, the individual must meet one of the following 4 criteria:

A fasting plasma glucose (FPG) of ≥126 mg/dL (7.0 mmol/L). Fast no caloric intake for at least 8 hours.

A 2-hour plasma glucose (PG) of ≥200 mg/dL (11.1 mmol/L) during an oral glucose tolerance test (OGTT). The test should be performed as described by the World Health Organization (WHO), using a glucose load containing the equivalent of 75 g anhydrous glucose dissolved in water.

A hemoglobin A1C (HbA1c) of ≥6.5% (48 mmol/mol). The test should be performed in a laboratory using a method that is National Glycohemoglobin Standardization Program (NGSP) certified and standardized to the Diabetes Control and Complications Trial (DCCT) assay.

In a patient with classic symptoms of hyperglycemia or hyperglycemic crisis, a random PG of ≥200 mg/dL (11.1 mmol/L).

For the diagnosis of clinical Type 1 diabetes (T1D), the ADA suggests that plasma blood glucose rather than HbA1C should be used to diagnose the acute onset of T1D in individuals with symptoms of hyperglycemia.

According to ADA, a patient with classic symptoms, measurement of plasma glucose is sufficient to diagnose clinical diabetes (symptoms of hyperglycemia or hyperglycemic crisis plus a random plasma glucose ≥200 mg/dL [11.1 mmol/L]). In these cases, knowing the plasma glucose level is critical because, in addition to confirming that symptoms are due to diabetes, it will inform management decisions. Some providers may also want to know the HbA1C to determine how long a patient has had hyperglycemia. In addition, T1D, previously called “insulin-dependent diabetes” or “juvenile-onset diabetes,” accounts for 5-10% of diabetes and is due to cellular-mediated autoimmune destruction of the pancreatic β-cells. Autoimmune markers include islet cell autoantibodies and autoantibodies to GAD (GAD65), insulin, the tyrosine phosphatases IA-2 and IA-2 B, and ZnT8. T1D is defined by the presence of one or more of these autoimmune markers.

In some embodiments, the diagnosis of T1D is made with the use of a continuous glucose monitoring system (CGM) revealing high sensor average glucose levels (>=110 mg/dL), or high variability of glycemia (CV >=15), or less time in range (>=10% of the time above 140 mg/dL).

In some embodiments, the patient diagnosed with clinical T1D has a positive result on testing for at least one of the following T1D-related autoantibodies: Glutamic acid decarboxylase 65 (GAD65) autoantibodies, Islet antigen 2 (IA-2) autoantibodies, Zinc transporter 8 (ZnT8) autoantibodies Islet cell cytoplasmic autoantibodies (ICA) or Insulin autoantibodies (if testing obtained within the first 14 days of insulin treatment embodiments, the presence of the autoantibodies is detected by ELISA, electrochemoluminescence (ECL), radioassay (see, e.g., Yu et al., 1996, J. Clin. Endocrinol. Metab. 81:4264-4267), agglutination PCR (Tsai et al, ACS Central Science 2016 2 (3), 139-147) or by any other method for immunospecific detection of antibodies described herein or as known to one of ordinary skill in the art.

It is recognized that β cells continue to be lost following T1D diagnosis. To maximize the effect of β cell preservation in patients with a recoverable level endogenous insulin production, the patients to be treated ion some embodiments are within 6 weeks from T1D diagnosis and have a peak C-peptide level of ≥0.2 pmol/mL during a mixed meal tolerance test (MMTT).

In some embodiments, the methods provided herein reduces, prevents or delays the need for administration of exogenous insulin to the patients.

β-cell function prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HA1c) as the standard for evaluation of blood glucose control (DCCT, 1993, N. Engl. J. Med. 329:977-986). Alternatively, characterization of daily insulin needs, C-peptide levels/response, hypoglycemic episodes, and/or FPIR may be used as markers of β-cell function or to establish a therapeutic index (See Keymeulen et al., (2005) N Engl J Med. 352:2598-608; Herold et al., (2005) Diabetes 54:1763-9; U.S. Pat. Appl. Pub. No. 2004/0038867 A1; and Greenbaum et al., (2001) Diabetes 50:470-6, respectively). For example, FPIR is calculated as the sum of insulin values at 1 and 3 minutes post IGTT, which are performed according to Islet Cell Antibody Register User's Study protocols (see, e.g., Bingley et al., (1996) Diabetes 45:1720-8 and McCulloch et al., (1993) Diabetes Care 16:911-5).

In some embodiments, the effective amount of the anti-CD3 antibody such as teplizumab comprises a 10 to 14 day course of subcutaneous (SC) injection or intravenous (IV) infusion or oral administration of the anti-CD3 antibody at a cumulative dose greater than 10,000 micrograms/meter squared (μg/m²).

In some embodiments, the effective amount of the anti-CD3 antibody such as teplizumab comprises a 10 to 14 day course of subcutaneous (SC) injection or intravenous (IV) infusion or oral administration of the anti-CD3 antibody at from about 9,500 to about 14,000 μg/m², from about 9,500 to about 13,500 μg/m², from about 9,500 to about 13,000 μg/m², from about 9,500 to about 12,500 μg/m², from about 9,500 to about from about 9,500 to about 11,500 μg/m², from about 9,500 to about 11,000 μg/m², from about 9,500 to about 10,500 μg/m², from about 9,500 to about 10,000 μg/m². In some embodiments, the effective amount of the anti-CD3 antibody such as teplizumab comprises a 10 to 14 day course of subcutaneous (SC) injection or intravenous (IV) infusion or oral administration of the anti-CD3 antibody at about 10,000 μg/m², 10,500 μg/m², 11,000 μg/m², 11.500 μg/m², 12,000 μg/m², 12,500 μg/m², 13,000 μg/m², 13,500 μg/m², or 14,000 μg/m². In some embodiments, the anti-CD3 antibody is or comprises teplizumab.

In some embodiments, the effective amount comprises a 12-day course to 14-day course of subcutaneous intravenous (IV) infusion of the anti-CD3 antibody such as teplizumab at 106-850 micrograms/meter squared (μg/m²). In some embodiments, the total dosage over the duration of the regimen is about 14000 μg/m², 13500 μg/m², 13000 μg/m², 12500 μg/m², 12000 μg/m², 11500 μg/m², 11000 μg/m², 10500 μg/m², 10000 μg/m², 9500 μg/m², 9000 μg/m², 8000 μg/m², 7000 μg/m², 6000 μg/m², and may be less than 5000 μg/m², 4000 μg/m², 3000 μg/m², 2000 μg/m², or 1000 μg/m². In some embodiments, the total dosage over the duration of the regimen is from about 9030 μg/m² to about 14000 μg/m², about 9030 μg/m² to about 13500 μg/m², about 9000 μg/m² to about 13000 μg/m², about 9000 μg/m² to about 12500 μg/m², about 9000 μg/m² to about 12000 μg/m², about 9000 μg/m² to about 11500 μg/m², about 9000 μg/m² to about 11000 μg/m², about 9000 μg/m² to about 10500 μg/m², about 9000 μg/m² to about 10000 μg/m², about 9000 μg/m² to about 9500 μg/m². In some embodiments, the total dosage over the duration of the regimen is from about 9030 μg/m² to about 14000 μg/m², about 9030 μg/m² to about 13500 μg/m², about 9030 μg/m² to about 13000 μg/m², about 9030 μg/m² to about 12500 μg/m², about 9030 μg/m² to about 12000 μg/m², about 9030 μg/m² to about 11500 μg/m², from about 9030 g/m² to about 11000 μg/m², about 9030 μg/m² to about 10500 μg/m², about 9030 μg/m² to about 10000 μg/m², about 9030 μg/m² to about 9500 μg/m².

Without being bound by the theory, cumulative doses above about 9,000 μg/m² of teplizumab are expected to have comparable efficacy in terms of C-peptide preservation as shown for about 9,000 mg. That is because the exposure/response curve surprisingly reaches a plateau above which increasing doses do not result in increased efficacy. The evaluation of C-peptide preservation was performed utilizing the Protégé study data. Model-predicted teplizumab AUCs versus change from baseline in C-peptide were plotted and an Emax analysis was performed. These data demonstrate that an Emax model describes the relationship between teplizumab exposure and change in C-peptide at 2 years. At teplizumab AUC levels greater than about 1500 ng*hr/mL (below the lowest AUC pre about 9,000 μg/m² dose, of 1,789 ng*hr/mL) no additional improvement in C-peptide with increased teplizumab exposure was observed. Therefore, these data suggest that doses above about 9,000 mg of teplizumab would have comparable efficacy in terms of C-peptide preservation as shown for about 9,000 mg.

In some embodiments, the effective amount comprises a 12-day course IV infusion of teplizumab at a first dose of 106 μg/m² teplizumab on day 1, a second dose of 425 μg/m² teplizumab on day 2, and one dose of 850 μg/m² on each of days 3-12.

In some embodiments, the effective amount comprises a 12-day course IV infusion of teplizumab at a first dose of 211 μg/m² teplizumab on day 1, a second dose of 423 μg/m² teplizumab on day 2, and one dose of 840 μg/m² on each of days 3-12.

In some embodiments, the effective amount comprises a 12-day course IV infusion of teplizumab at a first dose of approximately 100 μg/m² teplizumab on day 1, a second dose of approximately 400 μg/m² teplizumab on day 2, a third dose of approximately 850 μg/m² on day 3, and approximately 1,200 μg/m² on each of days 4-12.

In some embodiments, the effective amount comprises a 12-day course IV infusion of teplizumab at a first dose of approximately 100 μg/m² teplizumab on day 1, a second dose of approximately 400 μg/m² teplizumab on day 2, a third dose of approximately 850 μg/m² on day 3, and approximately 1,300 μg/m² on each of days 4-12.

In some embodiments, the effective amount comprises a 12-day course IV infusion of teplizumab at a first dose of approximately 100 μg/m² teplizumab on day 1, a second dose of approximately 400 μg/m² teplizumab on day 2, a third dose of approximately 850 μg/m² on day 3, and approximately 1,400 μg/m² on each of days 4-12.

In some embodiments, the effective amount comprises a 12-day course IV infusion of teplizumab at a first dose of approximately 200 μg/m² teplizumab on day 1, a second dose of approximately 400 μg/m² teplizumab on day 2, a third dose of approximately 850 μg/m² on day 3, and approximately 1,200 μg/m² on each of days 4-12.

In some embodiments, the effective amount comprises a 12-day course IV infusion of teplizumab at a first dose of approximately 200 μg/m² teplizumab on day 1, a second dose of approximately 400 μg/m² teplizumab on day 2, a third dose of approximately 850 μg/m² on day 3, and approximately 1,300 μg/m² on each of days 4-12.

In some embodiments, the effective amount comprises a 12-day course IV infusion of teplizumab at a first dose of approximately 200 μg/m² teplizumab on day 1, a second dose of approximately 400 μg/m² teplizumab on day 2, a third dose of approximately 850 μg/m² on day 3, and approximately 1,400 μg/m² on each of days 4-12.

In some embodiments, the method comprises administering to the subject a 14-day course IV infusion at about 60 μg/m², about 125 μg/m², about 250 μg/m², and about 500 μg/m², on days 1-4, respectively, and a dose of about 1,000 μg/m² on each of days 5-14. In some embodiments, the cumulative dose is about 10,935 μg/m².

In some embodiments, the method comprises administering to the subject a 14-day course IV infusion at about 60 μg/m², about 125 μg/m², about 250 μg/m², and about 500 μg/m², on days 1-4, respectively, and a dose of about 1,030 μg/m² on each of days 5-14. In some embodiments, the cumulative dose is about 11,235 μg/m².

In some embodiments, the method comprises administering to the subject a 14-day course IV infusion at about 100 μg/m², about 425 μg/m², about 850 μg/m², and about 850 μg/m², on days 1-4, respectively, and a dose of about 1,000 μg/m² on each of days 5-14. In some embodiments, the cumulative dose is about 12,225 μg/m².

In some embodiments, the method comprises administering to the subject a 14-day course IV infusion at about 65 μg/m², about 125 μg/m², about 250 μg/m², and about 500 μg/m², on days 1-4, respectively, and a dose of about 1,070 μg/m² on each of days 5-14. In some embodiments, the cumulative dose is about 11,640 μg/m².

In some embodiments, the method comprises administering to the subject a 14-day course IV infusion at about 65 μg/m², about 125 μg/m², about 250 μg/m², and about 500 μg/m², on days 1-4, respectively, and a dose of about 1,030 μg/m² on each of days 5-14. In some embodiments, the cumulative dose is about 11,240 μg/m².

Provided herein is a dosing regimen comprising two or more courses of dosing with an anti-CD3 antibody such as teplizumab comprising a first course of dosing at week 1 and second course of dosing at week 26. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 9000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 9500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, days or 14 days, with a cumulative teplizumab dose of 10000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 10500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 11000 μg/m² for each course In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 11500 μg/m² for each course of treatment. of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 12000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 12500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 13000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a cumulative teplizumab dose of 13500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 10-days to 14-days, for example 12 days or 14 days, with a teplizumab dose of 14000 μg/m² for each course of treatment. In some embodiments, the 12 days course has a 2-day ramp-up phase and a 10-day fixed-, maximal dosing period. In some embodiments, 106 μg/m² teplizumab is administered on day 1, 425 μg/m² teplizumab teplizumab is administered on day 2, and 850 μg/m² teplizumab is administered on each of days 3-12.

In other embodiments, the course of dosing can be repeated at 2 month, 4 month, 5 month, 6 month, 8 month, 9 month, 10 month, 12 month, 15 month, 18 month, 24 month, 30 month, or 36 month intervals. In some embodiments, efficacy of the treatment with the anti-CD3 antibody such as teplizumab is determined as described herein, or as is known in the art, at 2 months, 4 months, 5 month, 6 months, 9 months, 12 months, 15 months, 18 months, 24 months, 30 months, or 36 months subsequent to the previous treatment.

In some embodiments, a subject is administered one or more doses, for example 12 daily doses, of the anti-CD3 antibody such as teplizumab at about 5-1200 μg/m², for example, 106-850 μg/m² to treat, or slow the progression of or ameliorate one or more symptoms of T1D.

In some embodiments, the subject is administered a treatment regimen comprising two courses of daily doses of an effective amount of the anti-CD3 antibody such as teplizumab, wherein the course of treatment is administered over 2 days, 3 days, 4 days, 5 days, 6 days, 7 days, 8 days, 9 days, 10 days, 11 days, 12 days, 13 days or 14 days. In some embodiments, the treatment regimen comprises administering doses of the effective amount every day, every 2nd day, every 3rd day or every 4th day.

In some embodiments, a subject is administered a treatment regimen comprising one or more doses of a prophylactically effective amount of the anti-CD3 antibody such as teplizumab, wherein the prophylactically effective amount is 200 μg/kg/day, 175 μg/kg/day, 150 μg/kg/day, 125 μg/kg/day, 100 μg/kg/day, 95 μg/kg/day, 90 μg/kg/day, 85 μg/kg/day, 80 μg/kg/day, 75 μg/kg/day, 70 μg/kg/day, 65 μg/kg/day, 60 μg/kg/day, 55 μg/kg/day, 50 μg/kg/day, 45 μg/kg/day, 40 μg/kg/day, 35 μg/kg/day, 30 μg/kg/day, 26 μg/kg/day, 25 μg/kg/day, 20 μg/kg/day, 15 μg/kg/day, 13 μg/kg/day, 10 μg/kg/day, 6.5 μg/kg/day, 5 μg/kg/day, 3.2 μg/kg/day, 3 μg/kg/day, 2.5 μg/kg/day, 2 μg/kg/day, 1.6 μg/kg/day, 1.5 μg/kg/day, 1 μg/kg/day, 0.5 μg/kg/day, 0.25 μg/kg/day, 0.1 μg/kg/day, or 0.05 μg/kg/day; and/or wherein the prophylactically effective amount is 1200 μg/m²/day, 1150 μg/m²/day, 1100 μg/m²/day, 1050 μg/m²/day, 1000 μg/m²/day, 950 μg/m²/day, 900 μg/m²/day, 850 μg/m²/day, 800 μg/m²/day, 750 μg/m²/day, 700 μg/m²/day, 650 μg/m²/day, 600 μg/m²/day, 550 μg/m²/day, 500 μg/m²/day, 450 μg/m²/day, 400 μg/m²/day, 350 μg/m², μg/m²/day, 250 μg/m² day, 200 μg/m²/day, 150 μg/m²/day, 100 μg/m²/day, 50 μg/m²/day, 40 μg/m² day, 30 μg/m²/day, 20 μg/m²/day, 15 μg/m²/day, 10 μg/m²/day, or 5 μg/m²/day.

In some embodiments, the intravenous dose of 1200 μg/m² or less, 1150 μg/m² or less, 1100 μg/m² or less, 1050 μg/m² or less, 1000 μg/m² or less, 950 μg/m² or less, 900 μg/m² or less, 850 μg/m² or less, 800 μg/m² or less, 750 μg/m² or less, 700 μg/m² or less, 650 μg/m² or less, 600 μg/m² or less, 550 μg/m² or less, 500 μg/m² or less, 450 μg/m² or less, 400 μg/m² or less, 350 μg/m² or less, 300 μg/m² or less, 250 μg/m² or less, 200 μg/m² or less, 150 μg/m² or less, 100 μg/m² or less, 50 μg/m² or less, 40 μg/m² or less, 30 μg/m² or less, 20 μg/m² or less, 15 μg/m² or less, 10 μg/m² or less, or 5 μg/m² or less of the anti-CD3 antibody such as teplizumab is administered over about 24 hours, about 22 hours, about 20 hours, about 18 hours, about 16 hours, about 14 hours, about 12 hours, about 10 hours, about 8 hours, about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of type 1 diabetes. In some embodiments, the total dosage over the duration of the regimen is a total of less than about 14000 μg/m², 13500 μg/m², 13000 μg/m², 12500 μg/m², 12000 μg/m², 11500 μg/m², 11000 μg/m², 10500 μg/m², 10000 μg/m², 9500 μg/m², 9000 μg/m², 8000 μg/m², 7000 μg/m², 6000 μg/m², and may be less than 5000 μg/m², 4000 μg/m², 3000 μg/m², 2000 μg/m², or 1000 μg/m². In some embodiments, the daily dosage administered in the regimen is from about 100 μg/m² to about 200 μg/m², about 100 μg/m² to about 500 μg/m², about 100 μg/m² to about 1000 μg/m², or about 500 μg/m² to about 1000 μg/m².

In some embodiments, the dose escalates over the first three, first ¼ of the doses (e.g., over the first 3 days of a 12-day regimen of one dose per day) of the treatment regimen until the daily effective amount of the anti-CD3 antibody such as teplizumab is achieved. In some embodiments, a subject is administered a treatment regimen comprising one or more doses of an effective amount of the anti-CD3 antibody such as teplizumab, wherein the effective amount is increased by, e.g., 0.01 μg/kg, 0.02 μg/kg, 0.04 μg/kg, 0.05 μg/kg, 0.06 μg/kg, 0.08 g/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg each day; or increased by, e.g., 100 μg/m², 150 μg/m², 200 μg/m², 250 μg/m², 300 μg/m², 350 μg/m², 400 μg/m², 450 μg/m², 500 μg/m², 550 μg/m², 600 μg/m², or 650 μg/m², each day as treatment pro embodiments, a subject is administered a treatment regimen comprising one or more doses of an effective amount of the anti-CD3 antibody such as teplizumab, wherein the effective amount is increased by a factor of 1.25, a factor of 1.5, a factor of 2, a factor of 2.25, a factor of 2.5, or a factor of 5 until the daily effective amount of the anti-CD3 antibody such as teplizumab is achieved.

In some embodiments, a subject is intramuscularly administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab to treat or ameliorate one or more symptoms of T1D.

In some embodiments, a subject is subcutaneously administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab to treat or ameliorate one or more symptoms of T1D.

In some embodiments, a subject is intravenously administered one or more doses of a 100 μg/kg or less, preferably 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab to treat or ameliorate one or more symptoms of T1D. In some embodiments, the intravenous dose of 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 teplizumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to treat or ameliorate one or more symptoms of T1D.

In some embodiments, a subject is orally administered one or more doses of a 100 μg/kg or less, preferably 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab to treat or ameliorate one or more symptoms of T1D. In some embodiments, the oral dose of 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.2 μg/kg or less of the anti-CD3 antibody such as teplizumab is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to treat or ameliorate one or more symptoms of T1D.

In some embodiments in which escalating doses are administered for the first days of the dosing regimen, the dose on day 1 of the regimen is 100-250 μg/m²/day, and escalates to the daily dose as recited immediately above by day 2, and 3. For example, on day 1, the subject is administered a dose of approximately 106 μg/m²/day, on day 2 approximately 425 μg/m²/day, and on subsequent days of the regimen (e.g., days 3-12) 850 μg/m²/day. In some embodiments, on day 1, the subject is administered a dose of approximately 211 μg/m²/day, on day 2 approximately 423 μg/m²/day, on day 3 and subsequent days of the regimen (e.g., days 3-12) approximately 840 μg/m²/day.

In some embodiments in which escalating doses are administered for the first days of the dosing regimen, the dose on day 1 of the regimen is about 5-150 μg/m²/day, preferably about 55-150 μg/m²/day, for example about 60-100 μg/m²/day and escalates to the daily dose as recited immediately above by day 3, 4, 5, 6 or 7. In some embodiments, on day 1, the subject is administered a dose of approximately 60 μg/m²/day, on day 2 approximately μg/m²/day, on day 3 approximately 250 μg/m²/day, on day 4 approximately 500 μg/m²/day and on subsequent days of the regimen (e.g., days 5-14) 1,000 μg/m²/day. In some embodiments, on day 1, the subject is administered a dose of approximately 60 μg/m²/day, on day 2 approximately 125 μg/m²/day, on day 3 approximately 250 μg/m²/day, on day 4 approximately 500 μg/m²/day and on subsequent days of the regimen (e.g., days 5-14) 1,030 μg/m²/day. In some embodiments, on day 1, the subject is administered a dose of approximately 100 μg/m²/day, on day 2 approximately 425 μg/m²/day, on day 3 approximately 850 μg/m²/day, on day 4 approximately 850 μg/m²/day and on subsequent days of the regimen (e.g., days 5-14) 1,000 μg/m²/day. In some embodiments, on day 1, the subject is administered a dose of approximately 60 μg/m²/day, on day 2 approximately 125 μg/m²/day, on day 3 approximately 250 μg/m²/day, on day 4 approximately 500 μg/m²/day and on subsequent days of the regimen (e.g., days 5-14) 1,070 μg/m²/day.

In some embodiments, to reduce the possibility of cytokine release and other adverse effects, the first 1, 2, or 3 doses or all the doses in the regimen are administered more slowly by intravenous administration. For example, a dose of 106 μg/m²/day may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, and about 22 hours. In some embodiments, the dose is administered by slow infusion over a period of, e.g., 20 to 24 hours. In some embodiments, the dose is infused in a pump, preferably increasing the concentration of antibody administered as the infusion progresses.

In some embodiments, a set fraction of the doses for the 106 μg/m²/day to 850 μg/m²/day regimen described above is administered in escalating doses.

In some embodiments, the anti-CD3 antibody such as teplizumab is not administered by daily doses over a number of days, but is rather administered by infusion in an uninterrupted manner over 4 hours, 6 hours, 8 hours, 10 hours, 12 hours, 15 hours, 18 hours, 20 hours, 24 hours, 30 hours or 36 hours. The infusion may be constant or may start out at a lower dosage for, for example, the first 1, 2, 3, 5, 6, or 8 hours of the infusion and then increase to a higher dosage thereafter. Over the course of the infusion, the patient receives a dose equal to the amount administered in the 5 to 20-day regimens set forth above. For example, a dose of approximately 150 μg/m², 200 μg/m², 250 μg/m², 500 μg/m², 750 μg/m², 1000 μg/m², 1500 μg/m², 2000 μg/m², 3000 μg/m², 4000 μg/m², 5000 μg/m², 6000 μg/m², 7000 μg/m², 8000 μg/m², 9000 μg/m², 9500 μg/m², 10000 μg/m², 10500 μg/m²,11000 μg/m², 11500 μg/m², 12000 μg/m², 12500 μg/m², 13000 μg/m², 13500 μg/m², can be administered. In particular, the speed and duration of the infusion is designed to minimize the level of free anti-CD3 antibody such as teplizumab in the subject after administration. In some embodiments, the level of free anti-CD3 antibody such as teplizumab should not exceed 200 ng/ml free antibody. In addition, the infusion is designed to achieve a combined T cell receptor coating and modulation of at least 50%, 60%, 70%, 80%, 90%, 95% or of 100%.

In some embodiments, the anti-CD3 antibody such as teplizumab is administered chronically to treat, or slow the progression, or ameliorate one or more symptoms of type 1 diabetes. For example, in some embodiments, a low dose of the anti-CD3 antibody such as teplizumab is administered once a month, twice a month, three times per month, once a week or even more frequently either as an alternative to the 6 to 14-day dosage regimen discussed above or after administration of such a regimen to enhance or maintain its effect. Such a low dose may be anywhere from 1 μg/m² to 100 μg/m², such as approximately 5 μg/m², 10 μg/m², 15 μg/m², 20 μg/m², 25 μg/m², 30 μg/m², 35 μg/m², 40 μg/m², 45 μg/m², or 50 μg/m².

In some embodiments, the subject may be re-dosed at some time subsequent to administration of the two course anti-CD3 antibody such as teplizumab dosing regimen, for example, based upon one or more physiological or biomarker parameters or may be done as a matter of course. Such redosing may be administered and/or the need for such redosing evaluated 2 months, 4 months, 6 months, 8 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years indefinitely.

In some embodiments, before and/or after (e.g., at a 1-6 month interval, or 2-5 month interval, or about 3 month interval) the administration of a 12-day course of teplizumab, the level (or relative amounts) of phenotypically exhausted T cells, such as TIGIT+KLRG1+CD8+CD3+ cells with respect to all CD3+ T cells is determined, for example by flow cytometry. In some embodiments, the level of the TIGIT+KLRG1+CD8+CD3+ T-cells can be monitored for example by flow cytometry. In some embodiments, an additional 12-day course of anti-CD3 antibody, such as teplizumab, is administered when the level of the TIGIT+KLRG1+CD8+CD3+ T-cells corresponds to (e.g., returns to) the baseline level. In some embodiments, the determining of TIGIT+KLRG1+CD8+CD3+ T-cells is about 3 months (or about 1-6 months) after the administration of the second 12-day course. In some embodiments, if the subject has more than about 10% TIGIT+KLRG1+CD8+ T-cells in all CD3+ T cells, the more annual. In some embodiments, if the subject has less than about 10% TIGIT+KLRG1+CD8+ T-cells in all CD3+ T cells, the monitoring can be every about 3-6 months.

In some embodiments, the re-dosing comprises administering additional (e.g., second, third, or beyond) 12-day to 14-day course(s) of teplizumab each at a total dose of more than about 9000 μg/m² as described herein. In some embodiments, the additional 12-day course of teplizumab comprises a first dose of 106 μg/m² teplizumab on day 1, a second dose of 425 μg/m² teplizumab on day 2, and one dose of 850 μg/m² on each of days 3-12, and wherein the total dose is approximately 9031 μg/m². In other embodiments the additional 12-day course of teplizumab comprises a first dose of 211 μg/m² teplizumab on day 1, a second dose of 423 μg/m² teplizumab on day 2, and one dose of 840 μg/m² on each of days 3-12, and wherein the total dose is approximately 9034 μg/m².

In some embodiments, the additional (e.g., second, third, or beyond) 12-day to 14-day course of anti-CD3 antibody, such as teplizumab, can be administered about 12 months to about 24 months after the administering of the prior 12-day to 14-day course, for example 12, 13, 14, 15, 16, 17, 19, 20, 21, 22, 23 or 24 months.

Provided herein are methods and teplizumab dosage regimen for lowering exogenous insulin use. In some embodiments, the anti-CD3 antibody such as teplizumab is administered to reduce exogenous insulin use that is lower by at least 0.08 U/kg/d (e.g. lower by 0.08 U/kg/d, 0.09 U/kg/d, 0.10 U/kg/d, 0.11 U/kg/d or more).

In some embodiments, subjects treated with anti-CD3 antibody, such as teplizumab, have lower exogenous insulin use (e.g. at least 0.08 U/kg/d less exogenous insulin use) as compared to pretreatment levels. In some embodiments, administration of anti-CD3 antibody, such as teplizumab, results in discontinuation of exogenous insulin use.

In some embodiments, the anti-CD3 antibody such as teplizumab is administered to achieve, or maintain a level of glycosylated hemoglobin (HA1 or HA1c) less than 8%, less than 7.5%, less than 7%, less than 6.5%, less than 6%, less than 5.5% or 5% or less. At the initiation of treatment, patients have a HA1 or HA1c level of less than 8%, less than 7.5%, less than 7%, less than 6.5%, less than 6%, or, more preferably, from 4%-6% (preferably measured in the absence of other treatment for diabetes, such as administration of exogenous insulin). Such patients preferably have retained at least 95%, 90%, 80%, 70%, 60%, 50%, 40% 30% or 20% of beta-cell function prior to initiation of treatment. In some embodiments, the administration of the anti-CD3 antibodies prevents damage, thereby slowing progression of the disease and reducing the need for insulin administration. In some embodiments, the methods of treatment provided herein result in a level of HA1 or HA1c is or less, 6% or less, 5.5% or less, or 5% or less 6 months, 9 months, 12 months, 15 months, 18 months, or 24 months after the previous treatment. In some embodiments, the administration of the anti-CD3 antibodies according to the methods provided herein decreases the average level of HA1 or HA1c in the patient by about 5%, about 10%, about 15%, about 20%, about 25%, about 30%, about 35%, about 40%, about 45%, about 50%, about 55%, about 60%, about 65% or about 70% as compared to pre-treatment levels at 6 months, 9 months, 12 months, 15 months, 18 months, or 24 months after the previous treatment. In some embodiments, the administration of the anti-CD3 antibodies according to the methods provided herein results in an average level of HA1 or HA1c in the patient that only increases by about 0.5%, about 1%, about 2.5%, about 5%, about 10%, about 15%, about 20%, about 25%, about 30%, about 35%, about 40%, about 45%, or about 50% as compared to pre-treatment levels at 6 months, 9 months, 12 months, 15 months, 18 months, or 24 months after the previous treatment.

In some embodiments, administration of the anti-CD3 antibodies, in particular teplizumab according to the methods provided herein slows the loss of β cells and/or preserves β cell function (as evidenced by e.g., C-peptide levels, episodes of hypo- or hyper-glycemia, time in range (of glycemia), insulin use, or other assessment method known in the art) over 12 months, 13 months, 14 months, 15 months, 16 months, 17 months, 18 months, 19 months, 20 months, 21 months, 22 months, 2 months, 24 month or more in children and adolescents 8-17 years old who have been diagnosed with T1D in the previous 6 weeks. In some embodiments, administration of the anti-CD3 antibodies, in particular teplizumab according to the methods provided herein slows the loss of β cells and/or preserves β cell function over 18 months (78 weeks) in children and adolescents 8-17 years old who have been diagnosed with T1D in the previous 5 to 31 weeks.

In some embodiments, the subject is an adult. In some embodiments, the subject is a pediatric subject. In some embodiments, the pediatric subject is 7 years old or older. In some embodiments, the pediatric subject is 6 years old or older. In some embodiments, the pediatric subject is 5 years old or older. In some embodiments, the pediatric subject is 4 years old or older. In some embodiments, the pediatric subject is 3 years old or older. In some embodiments, the pediatric subject is 1 year old or older. In some embodiments, the pediatric subject is an infant.

Some embodiments relate to Teplizumab for use in a method of treating clinical type 1 diabetes (T1D), comprising administering to a subject in need thereof a 12-day course of the teplizumab at a total dose of more than about 9000 μg/m².

In some embodiments, the total dose is between about 9000 and about 9500 μg/m². In some embodiments, the total dose is between about 9000 and about 14000 μg/m².

In some embodiments, the 12-day course comprises a first dose of 106 μg/m² teplizumab on day 1, a second dose of 425 μg/m² teplizumab on day 2, and one dose of 850 μg/m² on each of days 3-12, and wherein the total dose is approximately 9031 μg/m².

In some embodiments, the 12-day course comprises a first dose of 211 μg/m² teplizumab on day 1, a second dose of 423 μg/m² teplizumab on day 2, and one dose of 840 μg/m² on each of days 3-12, and wherein the total dose is approximately 9034 μg/m².

In some embodiments, the method can include administering a first and a second 12-day courses of teplizumab. In some embodiments, the first and the second 12-day courses are administered at about 1-6 months, about 2-5 months or about 3 months interval.

In some embodiments, the method can include administering to the subject in need thereof a third or more 12-day course of teplizumab, each course at a total dose of more than about 9000 μg/m².

In some embodiments, the third or more 12-day course of teplizumab comprises a first dose of 106 μg/m² teplizumab on day 1, a second dose of 425 μg/m² teplizumab on day 2, and one dose of 850 μg/m² on each of days 3-12, and wherein the total dose of each course is approximately 9031 μg/m².

In some embodiments, the third or more 12-day course of teplizumab comprises a first dose of 211 μg/m² teplizumab on day 1, a second dose of 423 μg/m² teplizumab on day 2, and one dose of 840 μg/m² on each of days 3-12, and wherein the total dose of each course is approximately 9034 μg/m².

In some embodiments, the third or more 12-day course of teplizumab is administered at about a 12 month to about a 24-month interval.

In some embodiments, the method can further include determining, after the administration of each 12-day course, a baseline of a level of TIGIT+KLRG1+CD8+ cells with respect to all CD3+ T cells, monitoring the level of the TIGIT+KLRG1+CD8+CD3+ T-cells and administering an additional 12-day course of teplizumab when the level of the TIGIT+KLRG1+CD8+CD3+ T-cells returns to the baseline level. In some embodiments, the determining of TIGIT+KLRG1+CD8+CD3+ T-cells is by flow cytometry. In some embodiments, the monitoring of TIGIT+KLRG1+CD8+CD3+ T-cells is by flow cytometry. In some embodiments, the determining of TIGIT+KLRG1+CD8+CD3+ T-cells is about 1-6 months, about 2-5 months, or about 3 months after the administration of each 12-day course.

In some embodiments, if the subject has more than about 10% TIGIT+KLRG1+CD8+ T-cell in all CD3+ T cells, subsequent monitoring is annual. In some embodiments, if the subject has less than about 10% TIGIT+KLRG1+CD8+ T-cells in all CD8+ T cells, subsequent monitoring is every about 3-6 months.

In some embodiments, the subject in need thereof has been diagnosed with T1D within 6 weeks prior to the administrating step.

In some embodiments, the administrating step results in reduction by at least 10% of insulin use, HbA1c levels, hypoglycemic episodes, or combinations thereof as compared to pre-treatment levels.

In some embodiments, each dose is administered parenterally.

In some embodiments, each dose is administered by intravenous infusion.

In some embodiments, the subject in need thereof is about 8 to 17 years old.

In some embodiments, the subject in need thereof have a peak C-peptide level of ≥0.2 pmol/mL during a mixed meal tolerance test (MMTT).

In some embodiments, the subject receiving teplizumab has a higher mean C-peptide value compared with a control receiving placebo.

In some embodiments, the method further includes assessing the area under the time-concentration curve (AUC) of C-peptide following a mixed meal tolerance test (MMTT), at 78 weeks.

In some embodiments, the subject in need thereof has at least 20% of beta-cell function prior the administration of the first dose.

In some embodiments, the reduction of insulin use, HbA1c levels, hypoglycemic episodes, or combinations thereof is over a period of 12 months or more.

Some aspects relate to a method of treating clinical type 1 diabetes (T1D), comprising administering to a subject in need thereof a 12-day course of teplizumab at a total dose of more than about 9000 μg/m². Some aspects relate to teplizumab for use in a method of treating clinical type 1 diabetes (T1D), comprising administering to a subject in need thereof a 12-day course of the teplizumab at a total dose of more than about 9000 μg/m².

In some embodiments, a method of treating clinical type 1 diabetes (T1D) is provided comprising administering to a subject in need thereof a 12-day course of teplizumab at a total dose of from about 9000 to about 9500 μg/m². In some embodiments, a method of treating clinical type 1 diabetes (T1D) is provided comprising administering to a subject in need thereof a 12-day course of teplizumab at a total dose of from about 9000 to about 14000 μg/m².

### V. Exemplary Embodiments

Non-limiting exemplary embodiments of the present disclosure are further listed below.

1. A method of reducing exogenous insulin use in a subject in need thereof, the method comprising administering to the subject a 12-day to 14-day course of teplizumab at a total dose of from about 9000 μg/m² and about 14000 μg/m², wherein administration of teplizumab results in a reduction of exogenous insulin use by at least 0.08 U/kg/day.
2. The method of embodiment 1 wherein administration of teplizumab results in a reduction of exogenous insulin use by at least 0.08 U/kg/day at year 1.
3 The method of embodiment 1 wherein administration of teplizumab results in a reduction of exogenous insulin use by at least 0.10 U/kg/day at year 2.
4 The method of embodiment 1 comprising administering to the subject a 12-day course of teplizumab.
5. The method of embodiment 1 comprising administering to the subject a 14-day course of teplizumab.
6. The method of embodiment 1, comprising administering a first and a second 12-day to 14-day courses of teplizumab.
7. The method of embodiment 6, wherein the first and the second 12-day to 14-day courses are administered at about 6 months interval.
8 The method of embodiment 1, comprising administering a first and a second 12-day courses of teplizumab.
9. The method of embodiment 8, wherein the first and the second 12-day courses are administered at about 6 months interval.
10. The method of embodiment 1, wherein the subject in need thereof has stage 3 type I diabetes.
11. The method of any one of embodiments 1-10, the method comprising administering teplizumab by intravenous infusion.
12. The method of any one of embodiments 1-10, wherein the subject in need thereof is about 7.5 years old or older.
13. The method of any one of embodiments 1-10, wherein reduction of exogenous insulin use is over a period of 1 year or more.
14. Teplizumab for use in a method of reducing exogenous insulin use in a subject in need thereof, the method comprising administering to the subject a 12-day to 14-day course of teplizumab at a total dose of from about 9000 μg/m2 and about 14000 μg/m², administration of teplizumab results in a reduction of exogenous insulin use by at least 0.08 U/kg/day.

### EXAMPLES

In November 2022, teplizumab became the first drug approved to delay the onset of Stage 3 type 1 diabetes in adults and pediatric patients aged 8 years and older with Stage 2 type 1 diabetes based on data from the pivotal study, TN-10. In order to evaluate the effects of teplizumab on preserving endogenous insulin production, an integrated analysis of C-peptide data from 609 patients (375 teplizumab and 234 controls) from five clinical trials in Stage 3 was conducted. The primary outcome of the integrated analysis, change from baseline in stimulated C-peptide, was significantly improved at year 1 (average increase 0.08 pmol/ml, p<0.0001) and year 2 (average increase 0.12 pmol/ml, p<0.0001) after one or two courses of teplizumab. An analysis was also conducted on exogenous insulin use which showed an overall reduction at year 1 of 0.08 U/kg/d (p<0.0001) and at year 2 of 0.10 U/kg/d (p=0.0001). An integrated safety analysis of five clinical trials which enrolled Stage 2 or Stage 3 patients including 1018 patients (approximately 1500 patient-years of follow-up for teplizumab-treated patients) was conducted. This analysis showed that the most common adverse events included lymphopenia, rash, and headache, the majority of which occurred during and after the first few weeks following teplizumab administration and generally resolved without intervention. These data confirm consistency in the preservation of beta cell function, as measured by C-peptide, across multiple clinical trials and a safety profile characterized by self-limited adverse events after one or two courses of teplizumab treatment.

### Introduction

Teplizumab was the first drug approved to change the progression of autoimmunity in type 1 diabetes and the first drug approved for delay of any autoimmune disease in patients prior to clinical onset. The approval followed decades of studies beginning with preclinical studies followed by six clinical trials including five in patients after clinical diagnosis (Stage 3) and one in patients prior to clinical diagnosis (Stage 2) who had 2 or more pancreatic islet autoantibodies and dysglycemia.

Teplizumab is a humanized IgG1 monoclonal antibody (mAb) that binds with high affinity to the epsilon chain of CD3 (CD38). Its complementarity determining region (CDR) is derived from Ortho Kung T3 (OKT3), the first mAb to be licensed for human use for acute solid graft rejection. OKT3 was humanized to minimize immunogenicity. Two Leu-to-Ala substitutions in the Fc region were introduced to minimize Fc-receptor binding 100- to 1,000-fold reduction in T-cell activation, T-cell proliferation, and cytokine release in human peripheral blood mononuclear cell cultures compared to OKT3.¹⁻⁵ Early studies of its mechanism showed that the partial agonism led to effects of CD8+ T cells that are integral to the autoimmune-mediated destruction of pancreatic beta cells.⁶⁻¹⁰

Teplizumab was initially investigated for treatment of acute transplant rejection and psoriatic arthritis.¹¹⁻¹⁴ At the same time, experiments in spontaneous and chemically induced diabetic mouse models provided evidence for reversal or prevention of autoimmune diabetes and immune tolerance since continuous administration, a requirement for previously studied immune therapies, was not required.¹⁵⁻¹⁷

The first randomized clinical trial in Stage 3 type 1 diabetes was a Phase 2 clinical study (Study 1), which evaluated the safety, tolerability, and efficacy of teplizumab given as a single 12- or 14-day course versus standard of care in patients with newly diagnosed Stage 3 diabetes.¹⁸ Efficacy was evaluated based on preservation of beta cell function by assessing C-peptide responses to a mixed meal tolerance test (MMTT). The success of this trial led to a randomized Phase 2 trial (AbATE [NCT00129259]) which administered a second course of teplizumab after 1 year in an effort to prolong the duration of response.¹⁸⁻²⁰ Subsequently, two Phase 3 trials (Protégé and Encore [NCT00385697 and NCT00920582]) testing three dosing regimens of teplizumab, administered over two courses 6 months apart, were conducted. These trials used a novel and unvalidated composite primary endpoint of exogenous insulin use and HbA1c. In an interim analysis, the Protégé study did not meet the primary endpoint which led to the termination and discontinuation of further clinical development.²¹ At the time these two studies were terminated, another Phase 2 trial (Delay [NCT00378508]) evaluated the effect of teplizumab in patients who were 4-12 months after type 1 diabetes diagnosis and had clinically significant levels of stimulated C-peptide (e.g., >0.2 pmol/ml).²² All five clinical studies showed promising C-peptide preservation.

The Stage 2 study, TN-10 (NCT01030861), was sponsored by National Institute of Diabetes and Digestive and Kidney Diseases (NIDDK) and conducted by Type 1 Diabetes TrialNet to evaluate whether a single course of teplizumab would delay or prevent the clinical diagnosis of type 1 diabetes in patients in early stage type 1 diabetes (Stage 2).²³ The study enrolled relatives of patients with type 1 diabetes, between 8 and 49 years of age, who had two or more islet autoantibodies and dysglycemia. The results showed a statistically significant delay in the median time to the onset of Stage 3 clinical type 1 diabetes of approximately 2 years compared to placebo patients indicating stabilization in the decline of beta cells leading to preservation of beta cell function with updated analysis extended delay in progression to 2.7 years.^{23,24}

While the results of the clinical studies support preservation of beta cell function as evaluated by C-peptide, a measure of endogenous insulin production, the individual studies had different protocol designs, including heterogeneous eligibility criteria and nature of the control groups, which may impact the overall interpretation of the results.²⁵

Therefore, the primary aim of this integrated analysis was to confirm the consistency of teplizumab effect on beta cell function as measured by stimulated C-peptide levels across the five clinical trials in patients with Stage 3 clinical type 1 diabetes. A secondary efficacy analysis was conducted on exogenous insulin use. A comprehensive review of the safety experience with teplizumab in both Stage 2 and Stage 3 type 1 diabetes patients was also evaluated.

### Methods

### 1. Clinical Trials

The designs of the six clinical trials have been previously published (Table 1).^{19,20,22,23,26} The dosing regimens evolved over time to accommodate pediatric patients and based on safety experience from a 14-day dosing regimen (Study 1:4 doses of escalation, 10 days full dose based on body weight) to 12-day (Study 1:2 days escalation with 10 days full dose based on body surface area [BSA]) to 14-day (AbATE, Delay, Protégé, Encore: 4 days escalation with 10 days full dose based on BSA). All patients in the trials received standard of care management of type 1 diabetes with insulin treatment. The investigators were advised to treat-to-target blood glucose based on recommendations from the American Diabetes Association. Specific algorithms for insulin administration were not given.

The majority of patients received the full 14-day regimen via intravenous infusion of teplizumab including 51 μg/m² on day 1, 103 μg/m² on day 2, 206 μg/m² on day 3, 413 μg/m² on day 4, and 826 μg/m² on days 5 to 14, for a cumulative dose of 9,034 μg/m². Patients were randomized to receive two treatment courses in Protégé, Encore (at randomization and at month 6), and AbATE (at randomization and at month 12), and a single course of treatment in Study 1 and Delay, with the option of a second course (open label) in the Delay study. The Protégé and Encore studies also investigated lower cumulative dose regimens (6-day [2426 μg/m²] or one-third dose 14-day regimen [2985 μg/m²]). The lower dosing regimens in Protégé and the second course in Delay were not included in the efficacy analysis but were included in the safety analysis.

### 2. Efficacy Analyses

a. Analysis of C-peptide Responses

All five studies included in the integrated efficacy analyses were conducted in Stage 3 type 1 diabetes patients who received the full 14-day regimen and had a randomized, placebo, or standard of care fixed-timeframe design (Table 1). The patients in these studies were enrolled within 6, 8, 12, and 12 weeks of type 1 diabetes diagnosis for Study 1, AbATE, Protégé and Encore, or 4-12 months from type 1 diabetes diagnosis for the Delay study. All patients had detectable levels of stimulated C-peptide. Study 1 and AbATE were open-label with randomization to study drug or standard of care, whereas Delay, Protégé, and Encore were double-blind, randomized, placebo-controlled. Study 1, AbATE, and Delay were conducted by academic investigators at sites in the US whereas Protégé and Encore were conducted by MacroGenics at sites located in the US, Eastern Europe, and India.

A one-stage integrated Intention-to-Treat (ITT) analysis was conducted using individual patient data from all five studies that included 1-year follow-up, and the three studies (Study 1, AbATE, and Protégé) that included 2-year follow-up. The ITT population included all patients who had been randomized to the 12- and 14-day teplizumab regimens in Study 1 and the full 14-day teplizumab regimen in AbATE, Protégé, Encore, and Delay, as well as patients randomized to standard of care or placebo (e.g., the control group) in all five studies (Table 2).

The primary endpoint was the change from baseline in C-peptide area under the curve (AUC). Baseline was defined as the C-peptide measurement obtained prior to the initial dose of teplizumab. The AUC in the 4-hour MMTT was divided by 240 or the last measured time for C-peptide and transformed (ln(AUC+1)). C-peptide was measured by radioimmunoassay (Study 1), TOSOH assay (AbATE and Delay), and chemiluminescent assay on the Immulite® 2000 (Protégé and Encore).

TABLE 1
Study Design and Inclusion Criteria for Clinical Trials Included in the Integrated Efficacy and Safety Analyses

| | Study 1* | AbATE*^{, †} | Protégé*^{, †} | Encore*^{, †} | Delay*^{, †} | TN-10^{†} |
|---|---|---|---|---|---|---|
| Stage of type 1 | 3 | 3 | 3 | 3 | 3 | 2 |
| diabetes | | | | | | |
| Integrated | Yes | Yes | Yes | Yes | Yes | No |
| Efficacy | | | | | | |
| Analyses* | | | | | | |
| Integrated | No | Yes | Yes | Yes | Yes | Yes |
| Safety | | | | | | |
| Analysis^{†} | | | | | | |
| Design | Randomized, | Randomized, | Randomized, | Randomized, | Randomized, | Randomized, |
| | open-label | open-label | double-blind | double-blind | double-blind | double-blind |
| Control group | Standard of care | Standard of care | Placebo | Placebo | Placebo | Placebo |
| Number of | Control: 21 | Control: 25 | Placebo: 98 | Placebo: 62 | Placebo: 27 | Placebo: 32 |
| patients | Teplizumab: 21 | Teplizumab: 52 | Teplizumab: 453 | Teplizumab: 192 | Teplizumab: 31 | Teplizumab: 44 |
| Teplizumab | 495 μg/kg over | 9034 μg/m² | 9034 μg/m² (Full) | 9034 μg/m² | 9034 μg/m² | 9034 μg/m² |
| dose/dosing | 14 days (n = 12) | over 14 days | over 14 days | over 14 days | over 14 days | over 14 days |
| schedule | or | | (n = 245) | (n = 63) | | |
| | 19,559 μg/m² | | 2985 μg/m² (⅓) | 2985 μg/m² (⅓) | | |
| | over 12 days | | over 14 days | over 14 days | | |
| | (n = 9) | | (n = 102) | (n = 66) | | |
| | | | 2426 μg/m² (6-day) | 2426 μg/m² | | |
| | | | over 6 days | over 6 days | | |
| | | | (n = 106) | (n = 63) | | |
| Regimen | 1 course: | 2 courses: | 2 courses: | 2 courses: | 1 course: | 1 course: |
| | Baseline | Baseline, | Baseline, | Baseline, | Baseline^{†} | Baseline |
| | | 12 months | 6 months | 6 months | | |
| Follow-up | Baseline, | Baseline, | Baseline, | Baseline, | Baseline, | Baseline, Months 3, |
| times | Months 6, 12, | Months 6, 12, | Day 140, | Day 140, | Months 6, 12 | 6, and every 6 |
| | 18, 24 | 18, 24 | Months 12, 18, 24 | Months 12, 18, 24 | | months thereafter |
| Stimulated | Primary, | Primary, | Secondary, | Secondary, | Primary, | Secondary, |
| C-Peptide | at 2 years | at 2 years | time not | time not | at 1 year | time not specified |
| endpoint | | | specified | specified | | |
| status | | | | | | |

Key Inclusion Criteria

| | | | | | | |
|---|---|---|---|---|---|---|
| Age at entry | 7.5-30 years | 8-30 years | 8-35 years | 8-35 years | 8-30 years | 8-45 years |
| Time of Stage | ≤6 weeks | ≤8 weeks | ≤12 weeks | ≤12 weeks | 4-12 months | Not applicable, |
| 3 type | | | | | | patients were |
| 1 diabetes | | | | | | enrolled with |
| diagnosis | | | | | | Stage 2 type 1 |
| to treatment | | | | | | diabetes |
| Auto- | anti-GAD65, | ICA, anti-GAD65, | anti-ICA512, IA-2, | anti-ICA512, IA-2, | ICA, anti-GAD65, | ICA, anti-GAD65, |
| antibodies* | anti-ICA512, IAA | anti-ICA512 | anti-GAD65, IAA | anti-GAD65, IAA | anti-ICA512 | anti-ICA512, IAA |
| Auto- | anti-GAD65, | ICA, anti-GAD65, | anti-ICA512, IA-2, | anti-ICA512, IA-2, | ICA, anti-GAD65, | ICA, anti-GAD65, |
| antibodies* | anti-ICA512, IAA | anti-ICA512 | anti-GAD65, IAA | anti-GAD65, IAA | anti-ICA512 | anti-ICA512, IAA |
| C-Peptide | ≥0.2 nmol/L | ≥0.2 nmol/L | Detectable | Detectable | ≥0.2 nmol/L | Not specified |
| level | | | | | | |
| Weight | N/A | ≥25 kg | ≥36 kg | ≥36 kg | ≥27.5 kg | ≥26 kg |

*Study 1, AbATE, Protégé, Encore, and Delay were included in the integrated efficacy analyses.
^{†}AbATE, Protégé, Encore, Delay, and TN-10 were integrated in the pooled safety analysis.
Abbreviations: GAD = glutamic acid decarboxylase; IA = islet antigen; IAA = insulin autoantibodies; ICA = islet cell autoantibodies.

TABLE 2
Baseline Demographic and Clinical Characteristics of Patients Included in the Integrated Efficacy Analyses

| | | | | | | | | | | | Pooled | |
|---|---|---|---|---|---|---|---|---|---|---|---|---|
| | Study 1 | | AbATE | | Protégé | | Encore | | Delay | | | Placebo/ |
| | TPZ | Control | TPZ | Control | TPZ | Placebo | TPZ | Placebo | TPZ | Placebo | TPZ | Control |
| | n = 21 | n = 21 | n = 52 | n = 25 | n = 207 | n = 98 | n = 63 | n = 62 | n = 32 | n = 28 | n = 375 | n = 234 |
| Age, years, | 13.9 | 14.9 | 12.7 | 12.3 | 18.8 | 18.2 | 17.3 | 18.4 | 12.7 | 11.9 | 16.9 | 16.6 |
| mean (SD) | (5.4) | (6.0) | (4.9) | (4.1) | (7.4) | (7.3) | (6.4) | (7.9) | (4.1) | (4.0) | (7.1) | (7.2) |
| Sex, % male | 61.9 | 61.9 | 53.8 | 64 | 62.8 | 62.2 | 60.3 | 66.1 | 56.3 | 60.7 | 60.5 | 63.2 |
| BMI | NA | NA | 19.52 | 19.99 | 21.17 | 20.69 | 21.25 | 20.96 | 19.30 | 19.95 | 20.77 | 20.59* |
| mean (SD) | | | (3.8) | (4.1) | (4.1) | (4.3) | (4.3) | (3.5) | (3.0) | (4.0) | (4.0) | (4.0) |
| HbA1c, %, | 8.98 | 8.05 | 7.43 | 7.7 | 8.32 | 8.19 | 8.09 | 8.40 | 6.4 | 7.14 | 8.18 | 8.18 |
| mean (SD) | (1.7) | (1.1) | (0.99) | (1.23) | (2.01) | (2.03) | (2.0) | (2.0) | (0.8) | (1.2 | (1.90) | (1.86) |
| C-Peptide AUC, | 0.49 | 0.48 | 0.72 | 0.67 | 0.65 | 0.65 | 0.68 | 0.58 | 0.62 | 0.59 | 0.66 | 0.61 |
| nmol/L, mean | (0.2) | (0.2) | (0.29) | (0.28) | (0.53) | (0.44) | (0.56) | (0.35) | (0.30) | (0.42) | (0.48) | (0.38) |
| (SD) | | | | | | | | | | | | |
| Insulin use, | 0.51 | 0.44 | 0.39 | 0.39 | 0.61 | 0.64 | 0.59 | 0.61 | 0.41 | 0.39 | 0.56 | 0.56 |
| U/kg/day, | (0.25) | (0.21) | (0.26) | (0.17) | (0.40) | (0.31) | (0.29) | (0.33) | (0.14) | (0.20) | (0.36) | (0.30) |
| mean (SD) | | | | | | | | | | | | |
| Time from T1D | <6 | <6 | 5.8 | 5.4 | 8.5 | 8.5 | 9.1 | 8.6 | 30.9 | 30.7 | 10.0 | 10.6 |
| diagnosis, weeks, | | | (1.2) | (1.3) | (2.6) | (2.6) | (2.3) | (2.4) | (10.7) | (10.5) | (7.5) | (8.6) |
| mean (SD) | | | | | | | | | | | | |

*Pooled value for BMI excludes Study 1 due to data not available.
Abbreviations: AUC = area under the curve; BMI = Body Mass Index; NA = Not Available; SD = standard deviation; TPZ = teplizumab.

Two analyses were performed on both observed data and imputed multiple imputation methods under “missing at random” and “missing not at random” assumptions. The change from baseline C-peptide was analyzed using analysis of covariance (ANCOVA). The ANCOVA included treatment as a main effect with covariates of age, baseline C-peptide, study, and study-by-treatment interaction. The study-by-treatment interaction term was included to assess whether there were differences in effect depending on individual study features and to adjust for these differences. The least square means (LSM) for the respective C-peptide change from baseline AUC treatment differences along with 95% confidence intervals (CIs) were estimated using results from the ANCOVA. The number of treatment courses and their timing were determined by the study and therefore were not included as separate covariates in the ANCOVA. Parameter estimates and 95% CIs were generated for the imputed datasets by averaging the estimated values for the 100 imputations conducted. All parameter estimates of differences in treatment LSMs and respective 95% CIs were presented in Forest plots.

b. Analysis of Exogenous Insulin Use

A second integrated analysis evaluated exogenous daily insulin use (U/kg/day) in the same studies. The average total daily insulin dose (all insulins) was used for this analysis. Individual patient data of insulin use were included from all five studies with 1 year of follow-up, and from Study 1, AbATE, and Protégé where 2-year data were available. ANCOVA was used to assess treatment differences in insulin use with covariates including baseline insulin use, treatment, age, study, and study-by-treatment interaction. Analyses were performed on both observed and imputed data.

### 3. Safety Analysis

All five studies included in the integrated safety analysis were conducted in Stage 2 (TN-10) or Stage 3 (AbATE, Protégé, Encore, and Delay) clinical trials using BSA-based dosing regimens (Supplemental Tables 1 and 3). Due to the inclusion of weight-based dosing in Study 1, safety data from this study was not included. Safety assessments included analyses of adverse events (AEs), mechanism-related adverse events that were considered adverse events of special interest (AESI), and serious adverse events (SAEs). The safety population was comprised of all patients in the three dosing regimens (full 14-day, 6-day, and one-third dose 14-day regimens) who received at least one dose of teplizumab or who were randomized to placebo or standard of care (e.g., the control group). Data are described using descriptive statistics. All analyses were performed using SAS software version 9.4.

### Results

### 1. Efficacy Analyses

a. Analysis of C-Peptide Responses

A total of 609 patients from 5 clinical trials were included in the ITT population for the efficacy analysis, 375 in the teplizumab group and 234 in the control group. In the teplizumab group, 88% of patients completed their respective study. Three (0.8%) patients in the teplizumab group withdrew early due to an adverse event, and 17 (4.5%) were lost to follow-up. No subjects in the control group withdrew early due to an adverse event and 6 (2.6%) were lost to follow-up.

Baseline demographic and clinical characteristics were similar between the treatment groups, but there were a number of differences between study cohorts (Table 2). The Protégé and Encore patients were older and had a longer time from diagnosis than Study 1 and AbATE. Patients in the Delay trial, by design, had a duration of 4-12 months since diagnosis.

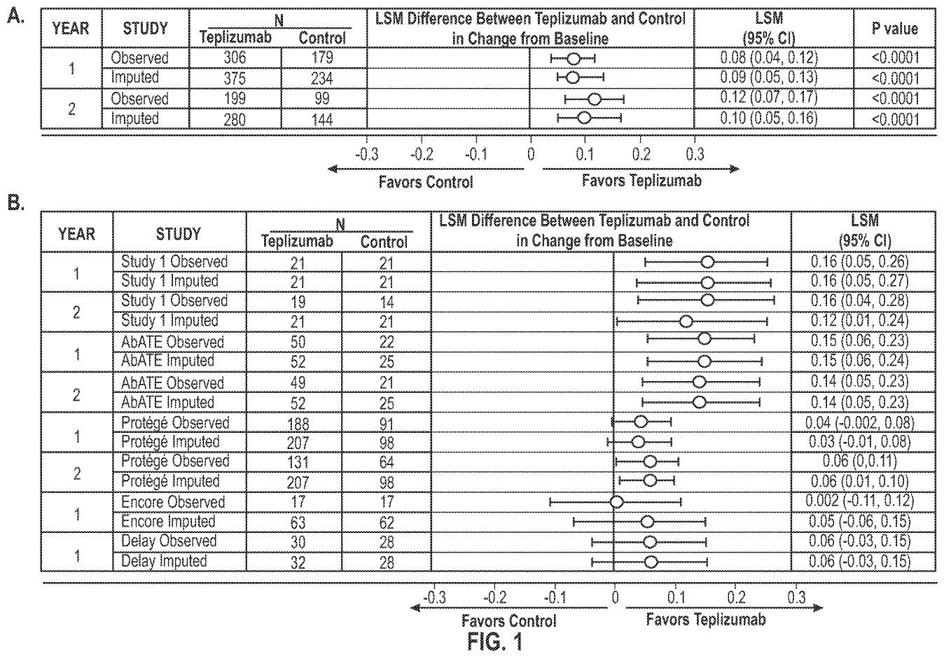

Forest plots of LSM differences and respective 95% CIs for changes from baseline C-peptide from the integrated analysis of the five studies are presented in FIG. 1, Panel A. These data show a highly statistically significant (p<0.0001) preservation of C-peptide with teplizumab treatment compared with placebo or standard of care for both the 1- and 2-year data (observed and imputed). For observed data, the difference in the treatment effect between the teplizumab and control groups was 0.08 (95% CI: 0.04, 0.12) pmol/mL at year 1 and 0.12 (95% CI: 0.07, 0.17) pmol/mL at year 2. For imputed data, the difference in the treatment effect between the teplizumab and control groups was 0.09 (95% CI: 0.05, 0.13) pmol/mL at year 1 and 0.10 (95% CI: 0.05, 0.16) pmol/mL at year 2.

The analysis by individual study and by year are shown in FIG. 1, Panel B. There was a significant improvement in C-peptide in each of the studies except Encore where there was a large amount of missing data due to the study's early termination. In each of the three studies with available 2-year data, teplizumab treatment significantly preserved C-peptide levels compared with placebo.

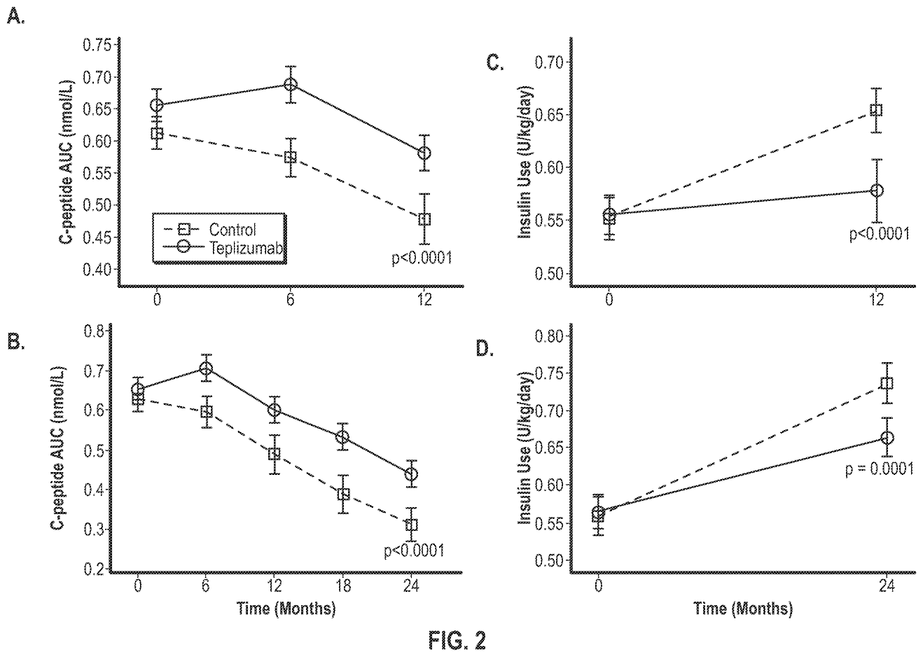

The C-peptide plots for 1- and 2-year data are shown in FIG. 2, Panels A and B, respectively. The C-peptide AUC levels at the 6-month timepoint showed an increased in the teplizumab group (an average increase of 5% and 8% in the 1-year and 2-year data, respectively) while they decreased in the control group. The C-peptide AUC decline from baseline at 1-year was 8% and 27% in the teplizumab and control groups, respectively (p<0.0001). For studies with 2-year data, the decline from baseline levels in teplizumab and controls, respectively (p<0.0001).

b. Integrated Analysis of Exogenous Insulin Use

Each clinical trial used a treat-to-target approach to diabetes management and therefore any effect of teplizumab treatment on endogenous beta cell function would be reflected by changes in the requirements for exogenous insulin.

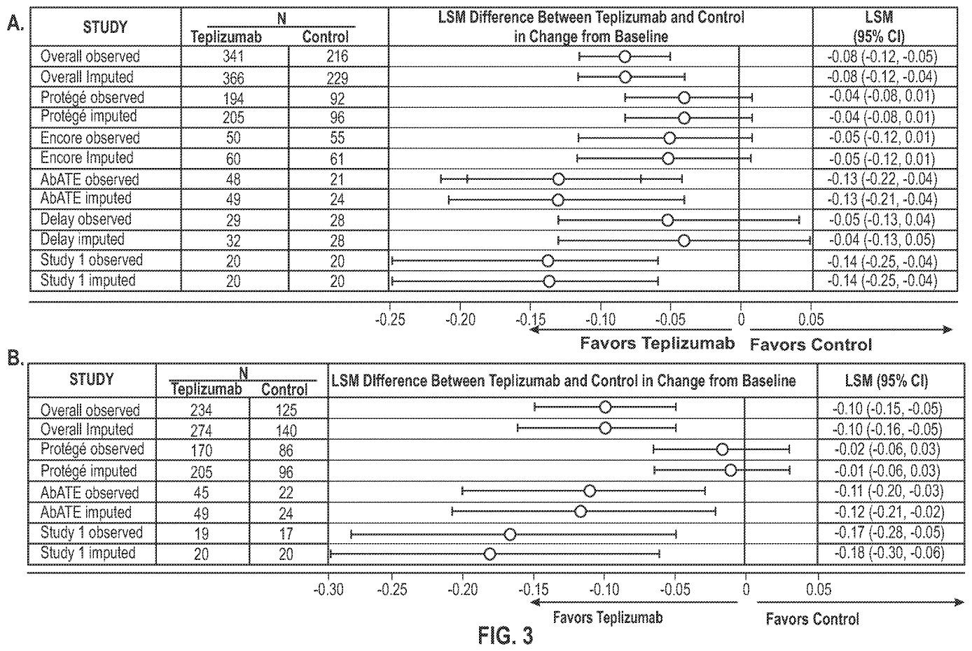

The Forest plots of the integrated analysis of insulin use showed statistically significant lower insulin use at 1 year (−0.08 U/kg/d [−0.12, −0.05]; p<0.0001) and 2 years (−0.10 U/kg/d [−0.15, −0.05]; p=0.0001) for teplizumab-treated patients compared with those in control groups FIG. 3, Panels A and B, respectively). In the individual studies, Study 1 and AbATE showed statistically significant lower insulin use for teplizumab-treated patients compared with control (p<0.0001 at 1 and 2 years, for both studies). However, point estimates across all studies favored lower insulin use in teplizumab-treated patients.

Insulin use over time are presented graphically in FIG. 2, Panels C and D. Baseline levels were similar in both groups. Significantly lower insulin use was observed at 1 year (0.58 vs 0.65 U/kg/day; p<0.0001) and for the three studies (AbATE, Protégé, and Encore) with 2-year data (0.66 vs 0.74 U/kg/day; p=0.0001). Insulin discontinuation data are only available for the Protégé study in which patients were not to discontinue insulin unless instructed to do so by the investigator. Despite being blinded to treatment, at 1 year 5.3% (11/207) of patients in the full dose 14-day teplizumab group were not taking insulin, compared with 0% (0/98) of controls (p=0.02). At year 2, 3 of these 11 patients remained off insulin whereas all control patients were still taking insulin (p<0.05).

### 2. Safety Analysis

a. Integrated Analysis of Safety

A total of 1,018 patients with Stage 2 and Stage 3 type 1 diabetes were included in the integrated safety analysis, 773 randomized to teplizumab treatment and 245 randomized to control (Table 3). There was approximately 1,500 patient-years of follow-up for the patients treated with teplizumab. The baseline demographic and clinical characteristics of this expanded group were similar between the treatment groups.

TABLE 3
Baseline Demographic and Clinical Characteristics of Patients Included in the Integrated Safety Analysis*

| | | | | | Protégé | | | | | | | Pooled | |
|---|---|---|---|---|---|---|---|---|---|---|---|---|---|
| | TN-10 | | AbATE | | OL | Protégé | | Encore | | Delay | | | Placebo/ |
| | TPZ | Control | TPZ | Control | TPZ | TPZ | Placebo | TPZ | Placebo | TPZ | Placebo | TPZ** | Control |
| | n = 44 | n = 32 | n = 52 | n = 25 | N = 38 | n = 415 | n = 98 | n = 192 | n = 62 | n = 32 | n = 28 | n = 773 | n = 245 |
| Age, | 19.2 | 17.5 | 12.7 | 12.3 | 16.5 | 18.4 | 18.2 | 17.7 | 18.4 | 12.7 | 11.9 | 17.6 | 16.9 |
| years, | (11.9) | (11.1) | (4.9) | (4.1) | (7.5) | (7.0) | (7.3) | (7.0) | (7.9) | (4.1) | (4.0) | (7.4) | (7.9) |
| mean | | | | | | | | | | | | | |
| (SD) | | | | | | | | | | | | | |
| Sex, | 56.8 | 53.1 | 53.8 | 64 | 78.9 | 63.6 | 62.2 | 67.7 | 66.1 | 56.3 | 60.7 | 64.0 | 62.0 |
| % male | | | | | | | | | | | | | |
| BMI | 21.94 | 22.08 | 19.52 | 19.99 | 20.20 | 21.38 | 20.69 | 21.24 | 20.96 | 19.30 | 19.95 | 21.11 | 20.78 |
| mean | (6.4) | (4.4) | (3.8) | (4.1) | (3.1) | (4.2) | (4.3) | (4.0) | (3.5) | (3.0) | (4.0) | (4.2) | (4.1) |
| (SD) | | | | | | | | | | | | | |

Abbreviations: BMI = Body Mass Index; OL = Open Label (Protégé had a single arm, open-label portion prior to the randomized study); SD = standard deviation; TPZ = teplizumab.
*The majority of patients received all doses of the study medication with 90.8% in the teplizumab group and 97.1% in the control group completing course 1 and 95.3% and 96.2%, respectively, completing course 2 (if that was part of the study design).
**18 Delay subjects initially randomized to the Placebo arm, who were later exposed to open-label teplizumab treatment in course 2 administration, are counted once for the demographics and subject exposure disposition but are counted as exposed to teplizumab for safety analyses.

A summary of adverse events (AE) is shown in Table 4. Almost always either treatment group experienced an AE, most of which were Grade 1 or Grade 2 in severity which typically occurred during and immediately following the dosing period. The majority of AEs resolved without intervention. AEs leading to permanent study drug discontinuation were reported in 14.3% and 3.7% of teplizumab and control groups, respectively. The most common reasons for discontinuation in the teplizumab group were laboratory investigations (7.7%; mainly liver enzyme elevations which met protocol-defined threshold for discontinuation), and blood and lymphatic disorders (3.2%; including neutropenia, thrombocytopenia, lymphopenia, and anemia).

TABLE 4
Summary of Adverse Events in Pooled Safety Population

| | Teplizumab | Control |
|---|---|---|
| | N = 791² | N = 245 |
| | n (%) | n (%) |
| Patients with at least 1 type of event¹ | | |
| AE | 787 (99.5) | 233 (95.1) |
| SAE | 98 (12.4) | 20 (8.2) |
| AE leading to treatment interruption | 88 (11.1) | 7 (2.9) |
| AE leading to treatment withdrawal | 113 (14.3) | 9 (3.7) |
| AE by CTCAE grade³ | | |
| Grade 1 | 749 (94.7) | 214 (87.3) |
| Grade 2 | 695 (87.9) | 146 (59.6) |
| Grade 3 | 467 (59.0) | 56 (22.9) |
| Grade 4 | 65 (8.2) | 5 (2.0) |
| Grade 5 | 2 (0.3) | 0 |
| Death | 24 (0.3) | 0 |

¹Patients who had more than 1 AE or SAE for a treatment were counted once.
²Delay patients initially randomized to the control arm, who were later also eligible for the open-label administration of teplizumab, are counted once for each treatment arm.
³Grading is unified based on existing grading; studies with CTCAE grading remain as coded for Protégé, Encore, and TN-10; grading in other studies (AbATE and Delay) was converted: mild to Grade 1, moderate to Grade 2, severe to Grade 3, SAE to Grade 4 and death to Grade 5.
⁴Both deaths occurred in the Protégé Study. There was one additional death in the Protégé Extension study.
Abbreviations: AE = adverse event, CTCAE = Common Terminology Criteria for Adverse Events, SAE = serious adverse event.

SAEs were reported in 12.4% and 8.2% of patients in the teplizumab and control groups, respectively, but most (76.7%) were considered not to be related to treatment. The most common SAEs were related to the underlying type 1 diabetes pathophysiology with the most notable being diabetic ketoacidosis (DKA) (n=18, 2.3% teplizumab; n=1, 0.4% control) and ketoacidosis (n=1, 0.1% teplizumab; n=0, control). These events were reported at an average of 10 months after the end of teplizumab treatment with a mean 11.9% near the time of event suggesting underlying poor glycemic control. SAEs of hypoglycemic events were only observed in Stage 3 patients and none occurred in Stage 2 patients. The rates of hypoglycemic unconsciousness and hypoglycemic coma were comparable in both groups.

The most common AEs reported in 10% or more of patients in either group are shown in Table 4. AEs reported at a higher frequency in the teplizumab group versus the control group were lymphopenia, leukopenia, neutropenia, decreased blood bicarbonate, and rash, all of which occurred during the dosing period and typically resolved within 4 weeks after the initiation of dosing. Lymphopenia was observed in approximately 80% of teplizumab-treated patients, with the nadir on Day 5 of treatment and resolved while dosing was continued, consistent with margination of lymphocytes and not depletion of the cells.

Cytokine release syndrome (CRS) was reported in 5.8% (46/791) and 1.2% (3/245) of teplizumab and control patients, respectively. CRS typically occurred during the first 3-5 days of dosing and resolved within 2 to 3 days of onset. Most (88%) events were Grade 1 or 2 in severity and treated with non-prescription medications.

The rates of infection were similar between the teplizumab-treated (53.0%) and control patients (52.7%) with serious infections occurring in 3.5% and 2% of teplizumab and control patients, respectively. New Epstein-Barr virus (EBV) infections were reported in 18 (2.3%) and 10 (4.1%) teplizumab and control patients, respectively. EBV reactivation was reported in 40 (5.1%) teplizumab-treated and 6 (2.4%) control patients. EBV viremia was detected in 25 (3.2%) and 3 (1.2%) teplizumab and control patients, respectively, 2 to 3 weeks after dosing, and was generally asymptomatic and resolved spontaneously without antiviral medications. New cytomegalovirus (CMV) infection was detected in 5 (0.6%) patients treated with teplizumab and 2 (0.8%) patients in the control group. Reactivation of CMV occurred in 4 (0.5%) teplizumab patients, none of which were associated with symptoms, and all cases resolved spontaneously without antiviral treatment.

Hypersensitivity reactions including anaphylaxis (0.1%), angioedema (0.3%), peripheral edema (1.6%), and urticaria (1.9%) were reported in teplizumab-treated patients; rash was observed in 48% and 15% of teplizumab and control patients, respectively.

### Discussion

For the first time, a disease-modifying therapy, TZIELD® (teplizumab-mzwv), has been approved to delay the onset of clinical Stage 3 type 1 diabetes. Teplizumab is thought to modify disease progression from Stage 2 to Stage 3 type 1 diabetes by preserving beta cell function. Over the past 20 years, five clinical trials of teplizumab in Stage 2 have demonstrated preservation of beta cell function, as measured by C-peptide levels. In order to evaluate consistency of beta cell preservation, an integrated analysis of C-peptide data from 5 clinical trials was conducted and supported by an analysis of exogenous insulin use. Review of the comprehensive safety experience was assessed by integrating data from 791 teplizumab-treated patients representing approximately 1,500 patient-years of follow-up.¹⁸⁻²³ This represents the most extensive data with a single agent in type 1 diabetes disease modification to date.

The integrated efficacy analysis showed that C-peptide levels were preserved to a significantly greater extent in patients treated with teplizumab compared with controls at 1- and 2-years post-treatment. Higher C-peptide levels, a measure of endogenous insulin production, were supported by reduced use of exogenous insulin.

It is important to note that the patients were heterogeneous in terms of age (a possible determinant of the rate of decline of C-peptide), location (North America, India, and Eastern Europe), clinical practices, and other patient characteristics such as pubertal status that may affect insulin sensitivity.²⁷ It is also likely that there were biologic differences in the autoimmune processes of patients since some patients were treated shortly after diagnosis while others were treated up to 1 year after diagnosis. Furthermore, different assays for C-peptide measurement were used. However, the same protocol for MMTTs was followed in all studies and the analyses involved changes in beta cell function from the baseline. Exploratory analysis of data from the Protégé study showed that a reduction in the decline in C-peptide response with teplizumab was greater in magnitude in patients diagnosed less than 6 weeks prior to randomization and also in patients from the US, whose metabolic parameters at baseline suggested less advanced disease than those in other regions.²¹ In the Delay study, which investigated teplizumab in patients diagnosed with Stage 3 type 1 diabetes within the previous 4 to 12 months, time from diagnosis did not significantly modify the treatment effect on C-peptide response.²² However, the magnitude of treatment effect between teplizumab and control patients on C-peptide response was less in that study than in studies where patients were treated with teplizumab within weeks of diagnosis. Thus, treatment soon after or even before the diagnosis of clinical disease may be needed for optimal responses to the drug. In Protégé and Delay, teplizumab was effective across all age groups, but younger patients treated with teplizumab had better C-peptide response than older patients. Data from these trials have been used in the design of the PROTECT study (NCT03875729),²⁸ which is evaluating teplizumab in patients 8 to 17 years of age with newly diagnosed Stage 3 type 1 diabetes who have evidence of residual beta cell function. The result of the analysis illustrates a significant difference in C-peptide preservation, across multiple studies, supporting the consistency of the effects of teplizumab treatment across and within studies suggest that its biologic effects are robust, reproducible, and not unique to a specific study or patient characteristics.

The safety profile of teplizumab was characterized by mild to moderate adverse events which were self-limited. CRS was observed in 5.8% and is likely attributable to the partial agonistic effect of teplizumab.^{1,29} Higher rates of serious infections (3.5% teplizumab vs 2% control) were observed although overall rates of infections were similar between treatment groups. The majority of teplizumab-treated patients developed lymphopenia (~80%) which resolved even while dosing continued and without treatment interruption.^{30,31} Importantly, the development of transient lymphopenia did not appear to be associated with an overall increased risk of infection. While not included in the integrated analysis, 7-year follow-up of patients in the AbATE study showed no increased risk of infections and no malignancies were observed.³² Previous preclinical studies and analyses of cells from teplizumab-treated patients have suggested that this partial agonist signal may lead to partial exhaustion of CD8+ T cells. Indeed, investigations of patients from the AbATE and rTN-10 study showed that the CD8+ TIGIT+KLRG1+ memory T cells from teplizumab-treated patients showed signs of exhaustion and produced lower levels of the inflammatory cytokines TNFα and IFNγ, which are associated with T-cell mediated beta cell death.²⁴ Since teplizumab targets activated effector cells and spares regulatory T cells and memory T cells against previously encountered pathogens, the preservation of beta cells is achieved without long-term impact on immune competence.³²

The integrated C-peptide analysis was unable to address whether the benefits of a second course of teplizumab are greater than those of a single course and future trials may be helpful to address this. The treat-to-target blood glucose study designs limited the ability to evaluate the effects of treatment on HbA1c levels. Nonetheless, in Study 1 and AbATE, improvement in HbA1c levels at some time points were observed.

In conclusion, the integrated efficacy analyses provide robust evidence for statistically significant preservation of beta cell function in patients with Stage 3 type 1 diabetes which was associated with the clinical benefit of reduced exogenous insulin requirement. These data provide the biological foundation of a disease-modifying therapy for type 1 diabetes and further support teplizumab as a promising treatment which addresses the underlying autoimmune pathogenesis of type 1 diabetes. The recent approve onset of Stage 3 type 1 diabetes in adults and pediatric patients aged 8 years and older with Stage 2 type 1 diabetes, built on the data presented herein, represents the first advance towards achieving the goal of developing disease-modifying therapies.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

1. Xu D, Alegre M L, Varga S S, et al. In vitro characterization of five humanized OKT3 effector function variant antibodies. Cell Immunol. Feb. 25, 2000; 200(1):16-26. doi:10.1006/cimm.2000.1617
2. Gaglia J, Kissler S. Anti-CD3 Antibody for the Prevention of Type 1 Diabetes: A Story of Perseverance. Biochemistry. Oct. 8, 2019; 58(40):4107-4111. doi:10.1021/acs.biochem.9b00707
3. Kuhn C, Weiner H L. Therapeutic anti-CD3 monoclonal antibodies: from bench to bedside. Immunotherapy. July 2016; 8(8):889-906. doi:10.2217/imt-2016-0049
4. L P. COBP. Prescribing Information Orthoclone OKT3. worldwideweb. drugs.com/pro/orthoclone-okt3.html
5. Masharani U B, Becker J. Teplizumab therapy for type 1 diabetes. Expert Opin Biol Ther. March 2010; 10(3):459-65. doi:10.1517/14712591003598843
6. Long S A, Thorpe J, DeBerg H A, et al. Partial exhaustion of CD8 T cells and clinical response to teplizumab in new-onset type 1 diabetes. Sci Immunol. November 2016; 1(5) doi:10.1126/sciimmunol.aai7793
7. Long S A, Thorpe J, Herold K C, et al. Remodeling T cell compartments during anti-CD3 immunotherapy of type 1 diabetes. Cell Immunol. September 2017; 319:3-9. doi:10.1016/j.cellimm.2017.07.007
8 Ablamunits V, Bisikirska B, Herold K C. Acquisition of regulatory function by human CD8(+) T cells treated with anti-CD3 antibody requires TNF. Eur J Immunol. October 2010; 40(10):2891-901. doi:10.1002/eji.201040485
9. Bisikirska B, Colgan J, Luban J, Bluestone J A, Herold K C. TCR stimulation with modified anti-CD3 mAb expands CD8+ T cell population and induces CD8+CD25+ Tregs. J Clin Invest. October 2005; 115(10):2904-13. doi:10.1172/jci23961
10. Lebastchi J, Deng S, Lebastchi A H, et al. Immune therapy and β-cell death in type 1 diabetes. Diabetes. May 2013; 62(5):1676-80. doi:10.2337/db12-1207
11. Woodle E S, Bluestone J A, Zivin R A, et al. Humanized, nonmitogenic OKT3 antibody, huOKT3 gamma (Ala-Ala): initial clinical experience. Transplant Proc. June 1998; 30(4):1369-70. doi:10.1016/s0041-1345(98)00278-4
12. Woodle E S, Xu D, Zivin R A, et al. Phase I trial of a humanized, Fc receptor nonbinding OKT3 antibody, huOKT3gamma1 (Ala-Ala) in the treatment of acute renal allograft rejection. Transplantation. Sep. 15, 1999; 68(5):608-16. doi:10.1097/00007890-199909150-00003
13. Utset T O, Auger J A, Peace D, et al. Modified anti-CD3 therapy in phase I/II clinical trial. J Rheumatol. September 2002; 29(9):1907-13.
14. Hering B J, Kandaswamy R, Harmon J V, et al. Transplantation of cultured islets from two-layer preserved pancreases in type 1 diabetes with anti-CD3 antibody. Am J Transplant. March 2004; 4(3):390-401. doi:10.1046/j.1600-6143.2003.00351.x
15. Hayward A R, Shreiber M. Neonatal injection of CD3 antibody into nonobese diabetic mice reduces the incidence of insulitis and diabetes. J Immunol. Sep. 1, 1989; 143(5):1555-9.
16. Chatenoud L, Thervet E, Primo J, Bach J F. Anti-CD3 antibody induces long-term remission of overt autoimmunity in nonobese diabetic mice. Proc Natl Acad Sci USA. Jan. 4, 1994; 91(1):123-7. doi:10.1073/pnas.91.1.123
17. Chatenoud L, Primo J, Bach J F. CD3 antibody-induced dominant self tolerance in overtly diabetic NOD mice. J Immunol. Mar. 15, 1997; 158(6):2947-54.
18. Herold K C, Hagopian W, Auger J A, et al. Anti-CD3 monoclonal antibody in new-onset type 1 diabetes mellitus. N Engl J Med. May 30, 2002; 346(22):1692-8. doi:10.1056/NEJMoa012864
19. Herold K C, Gitelman S E, Masharani U, et al. A single course of anti-CD3 monoclonal antibody hOKT3gamma1 (Ala-Ala) results in improvement in C-peptide responses and clinical parameters for at least 2 years after onset of type 1 diabetes. Diabetes. June 2005; 54(6):1763-9. doi:10.2337/diabetes.54.6.1763
20. Herold K C, Gitelman S E, Ehlers M R, et al. Teplizumab (anti-CD3 mAb) treatment preserves C-peptide responses in patients with new-onset type 1 diabetes in a randomized controlled trial: metabolic and immunologic features at baseline identify a subgroup of responders. Diabetes. November 2013; 62(11):3766-74. doi:10.2337/db13-0345
21. Sherry N, Hagopian W, Ludvigsson J, et al. Teplizumab for treatment of type 1 diabetes (Protégé study): 1-year results from a randomised, placebo-controlled trial. Lancet. Aug. 6, 2011; 378(9790):487-97. doi:10.1016/s0140-6736 (11) 60931-8
22. Herold K C, Bundy B N, Long S A, et al. An Anti-CD3 Antibody, Teplizumab, in Relatives at Risk for Type 1 Diabetes. N Engl J Med. Aug. 15, 2019; 381(7):603-613. doi:10.1056/NEJMoa1902226
23. Herold K C, Gitelman S E, Willi S M, et al. Teplizumab treatment may improve C-peptide responses in participants with type 1 diabetes after the new-onset period: a randomised controlled trial. Diabetologia. February 2013; 56(2):391-400. doi:10.1007/s00125-012-2753-4
24. Sims E K, Bundy B N, Stier K, et al. Teplizumab improves and stabilize function in antibody-positive high-risk individuals. Sci Transl Med. Mar. 3, 2021; 13(583) doi:10.1126/scitranslmed.abc8980
25. Jacobsen L M, Bundy B N, Greco M N, et al. Comparing Beta Cell Preservation Across Clinical Trials in Recent-Onset Type 1 Diabetes. Diabetes Technol Ther. December 2020; 22(12):948-953. doi:10.1089/dia.2020.0305
26. Herold K C, Gitelman S E, Ehlers M R, et al. Teplizumab (anti-CD3 mAb) treatment preserves C-peptide responses in patients with new-onset type 1 diabetes in a randomized controlled trial: metabolic and immunologic features at baseline identify a subgroup of responders. Diabetes. November 2013; 62(11):3766-74. doi:10.2337/db13-0345
27. Greenbaum C J, Beam C A, Boulware D, et al. Fall in C-peptide during first 2 years from diagnosis: evidence of at least two distinct phases from composite Type 1 Diabetes TrialNet data. Diabetes. August 2012; 61(8):2066-73. doi:10.2337/db11-1538
28. Recent-onset type 1 diabetes trial evaluating efficacy and safety of teplizumab (PROTECT): clinicaltrials.gov/ct2/show/NCT03875729
29. Alegre M L, Peterson L J, Xu D, et al. A non-activating “humanized” anti-CD3 monoclonal antibody retains immunosuppressive properties in vivo. Transplantation. Jun. 15, 1994; 57(11):1537-43.
30. Herold K C, Gitelman S, Greenbaum C, et al. Treatment of patients with new onset Type 1 diabetes with a single course of anti-CD3 mAb Teplizumab preserves insulin production for up to 5 years. Clin Immunol. August 2009; 132(2):166-73. doi:10.1016/j.clim.2009.04.007
31. Waldron-Lynch F, Henegariu O, Deng S, et al. Teplizumab induces human gut-tropic regulatory cells in humanized mice and patients. Sci Transl Med. Jan. 25, 2012; 4(118):118ra12. doi:10.1126/scitranslmed.3003401
32. Perdigoto A L, Preston-Hurlburt P, Clark P, et al. Treatment of type 1 diabetes with teplizumab: clinical and immunological follow-up after 7 years from diagnosis. Diabetologia. April 2019; 62(4):655-664. doi:10.1007/s00125-018-4786-9
33. Abdul-Rasoul M, Habib H, Al-Khouly M. “The honeymoon phase” in children with type 1 diabetes mellitus: frequency, duration, and influential factors. Pediatr Diabetes. 2006; 7(2):101-107.
34 Ablamunits V, Bisikirska B, Herold K C. Acquisition of regulatory function by human CD8+ T cells treated with anti-CD3 antibody requires TNF. Eur J Immunol. 2010; 40(10):2891-2901.
35. Agiostratidou G, Anhalt H, Ball D, et al. Standardizing clinically m measures beyond HbA1c for type 1 diabetes: a consensus report of the American Association of Clinical Endocrinologists, the American Association of Diabetes Educators, the American Diabetes Association, the Endocrine Society, JDRF International, The Leona M. and Harry B. Helmsley Charitable Trust, the Pediatric Endocrine Society, and the T1D Exchange. Diabetes Care. 2017; 40(12):1622-1630.
36. Akirav E M, Kushner J A, Herold K C. β cell mass and type 1 diabetes: Going, going, gone? Diabetes. 2008; 57:2883-2888.
37. American Diabetes Association. 12. Children and Adolescents: Standards of medical Care in Diabetes-2018. Diabetes Care. 2018; 41 (Suppl. 1):S126-S136.
38 Atkinson M A. Type 1 diabetes. Lancet. 2014; 383(9911):69-82
39 Bisikirska B, Colgan J, Luban J, Bluestone, J A, Herold K C. TCR stimulation with modified anti-CD3 mAb expands CD8+ T cell population and induces CD8+CD25+ Tregs. J Clin Invest. 2005; 115(10):2904-2913.
40. Bluestone J A, Herold K, and Eisenbarth G. Genetics, pathogenesis and clinical interventions in type 1 diabetes. Nature. 2010; 464(7293):1293-1300.
41. Boyle K D, Keyes-Elstein L, Ehlers M R, et al. Two- and four-hour tests differ in capture of C-peptide responses to a mixed meal in type 1 diabetes. Diabetes Care. 2016; 39:e76-78.
42. Bradley C, Loewenthal K, Woodcock A, et al. Development of the diabetes treatment satisfaction questionnaire (DTSQ) for teenagers and parents: the DTSQ-Teen and the DTSQ-Parent. Diabetologia. 2009; 52: (Suppl 1) S397, Abstract 1013.
43. Campbell-Thompson M, Fu A, Kaddis J S, et al. Insulitis and B-cell mass in the natural history of type 1 diabetes. Diabetes. 2016; 65:719-731.
44. Danne T, Nimri R, Battelino T, et al. International consensus on use of continuous glucose monitoring. Diabetes Care 2017; 40:1631-1640.
45. Diabetes Control and Complications Trial Research Group. Hypoglycemia in the Diabetes Control and Complications Trial. Diabetes. 1997; 46(2):271-286.
46. DiMeglio L A, Acerini C L, Codner E, et al. ISPAD Clinical Practice Consensus Guidelines 2018: Glycemic control targets and glucose monitoring for children, adolescents, and young adults with diabetes. Pediatr Diabetes. 2018 October; 19 Suppl 27:105-114.
47. Driscoll K A, Raymond J, Naranjo D, et al. Fear of hypoglycemia in children and adolescents and their parents with type 1 diabetes. Curr Diab Rep. 2016; 16(8):77.
48. European Medicines Agency. Ethical Considerations for Clinical T Products Conducted with the Paediatric Population. 2008. https://ec.europa.eu/health/sites/health/files/files/eudralex/vol-10/ethical_considerations_en.pdf. Accessed Sep. 27, 2018.
49. Fonolleda M, Murillo M, Vázquez F, et al. Remission phase in paediatric type 1 diabetes: new understanding and emerging biomarkers. Horm Res Paediatr. 2017; 88(5):307-315.
50. Gitelman S E, Gottlieb P A, Rigby M R, et al. Antithymocyte globulin treatment for patients with recent-onset type 1 diabetes: 12-month results of a randomised, placebo-controlled, phase 2 trial. Lancet Diabetes Endocrinol. 2013; 1(4):306-16.
51. Gonder-Frederick L, Nyer M, Shepard J A. Assessing fear of hypoglycemia in children with Type 1 diabetes and their parents. Diabetes Manag (Lond). 2011; 1(6):627-639.
52. Greenbaum C J, Beam C A, Boulware D et al. Fall in C peptide during first 2 years from diagnosis: evidence of at least two distinct phases from composite type 1 diabetes TrialNet data. Diabetes. 2012; 61:2066-2073.
53. Hagopian W, Ferry R J Jr, Sherry N et al, Protégé Trial Investigators. Teplizumab preserves C-peptide in recent-onset type 1 diabetes: two-year results from the randomized, placebo-controlled Protégé trial. Diabetes. 2013; 62(11):3901-8. doi:10.2337/db13-0236. Epub 2013 Jun. 25.
54 Helminen O, Pokka T, Tossavainen P, et al. Continuous glucose monitoring and HbAlc in the evaluation of glucose metabolism in children at high risk for type 1 diabetes mellitus. Diabetes Res Clin Pract. 2016; 120:89-96.
55. Herold K C, Hagopian W, Auger J A, Poumian-Ruiz E, Taylor L, Donaldson D, Gitelman S E, Harlan D M, Xu D, Zivin R A, Bluestone J A. Anti-CD3 monoclonal antibody in new-onset type 1 diabetes mellitus. N Engl J Med. 2002 May 30; 346(22):1692-1698.
56. Herold K C, Gitelman S E, Umest M et al. A single course of anti-CD3 monoclonal antibody hOKT3γ1 (Ala-Ala) results in improvement in C-peptide responses and clinical parameters for at least 2 years after onset of type 1 diabetes. Diabetes. 2005; 54:1-7.
57. Herold K C, Gitelman S E, Ehlers M R, et al. Teplizumab (anti-CD3 mAb) treatment preserves C-peptide responses in patients with new-onset type 1 diabetes in a randomized controlled trial: metabolic and immunologic features at baseline identify a subgroup of responders. Diabetes. 2013 (a); 62(11):3766-3774.
58. Herold K C, Gitelman S E, Willi S M, et al. Teplizumab treatment m; peptide responses in participants with type 1 diabetes after the new-onset period: a randomised controlled trial. Diabetologia. 2013 (b); 56(2):391-400.
59. Herold K C, Bundy B N, Long S A, et al. An Anti-CD3 Antibody, Teplizumab, in Relatives at Risk for Type 1 Diabetes. N Engl J Med. 2019 Jun. 9. doi: 10.1056/NEJMoa1902226. [Epub ahead of print]
60. Huo L, Harding J L, Peeters A, et al. Life expectancy of type 1 diabetic patients during 1997-2010: a national Australian registry-based cohort study. Diabetologia. 2016; 59(6):1177-85.
61. International Hypoglycaemia Study Group. Glucose Concentrations of Less Than 3.0 mmol/L (54 mg/dL) Should Be Reported in Clinical Trials: A Joint Position Statement of the American Diabetes Association and the European Association for the Study of Diabetes. Diabetes Care. 2016; dc162215.
62. Joint Commission. High Alert Medications and Patient Safety. Sentinel Event Alert. Issue 11. November 19,1999. https://www.jointcommission.org/assets/1/18/SEA_11.pdf. Accessed Aug. 21, 2018.
63. Karvonen M, Viik-Kajander M, Moltchanova E, et al. Incidence of childhood type 1 diabetes worldwide. Diabetes Mondiale (DiaMond) Project Group. Diabetes Care. 2000; 23(10):1516-1526.
64. Kuhtreiber W M, Washer S L, Hsu E, et al. Low levels of C-peptide have clinical significance for established Type 1 diabetes. Diabet Med. 2015 October; 32(10):1346-53.
65. Lachin J M, McGee P, Palmer J P; DCCT/EDIC Research Group. Impact of C-peptide preservation on metabolic and clinical outcomes in the Diabetes Control and Complications Trial. Diabetes. 2014; 63(2):739-748.
66. Laitinen O H, Honkanen H, Pakkanen O, et al. Coxsackievirus B1 is associated with induction of β-cell autoimmunity that portends type 1 diabetes. Diabetes. 2014; 63(2):446-455.
67. Lebastchi J, Deng S, Lebastchi A H, et al. Immune therapy and β-cell death in type 1 diabetes. Diabetes. 2013; 62(5):1676-1680.
68. Lin A, Northam E A, Werther G A, Cameron F J. Risk factors for decline in IQ in youth with type 1 diabetes over the 12 years from diagnosis/illness onset. Diabetes Care. 2015; 38:236-242.
69. Lind M, Svensson A M, Kosiborod M, et al. Glycemic control and excess mortality in type 1 diabetes. N Engl J Med. 2014; 371:1972-1982.
70. Long S A, Thorpe J, DeBerg H A, et al. Partial exhaustion of CD8 T response to teplizumab in new-onset type 1 diabetes. Sci. Immunol. 2016; 1(5):1-23.
71. Long S A, Thorpe J, Herold K C, et al. Remodeling T cell compartments during anti-CD3 immunotherapy of type 1 diabetes. Cell Immunol. 2017 September; 319:3-9.
72. Ludvigsson J, Carlsson A, Deli A, et al. Decline of C-peptide during the first year after diagnosis of Type 1 diabetes in children and adolescents. Diabetes Res Clin Pract. 2013; 100(2):203-209.
73. Masharani U B, Becker J. Teplizumab therapy for type 1 diabetes. Expert Opin Biol Ther. 2010; 10(3):459-465.
74. Matveyneko A V and Butler P C. Relationship between β cell mass and diabetes onset. Diabetes Obes Metab. 2008; 10:23-31.
75. Mittermayer F, Caveney E, De Oliveira C, et al. Addressing Unmet Medical Needs in Type 1 Diabetes: A Review of Drugs Under Development. Curr Diabetes Rev. 2017; 13(3):300-314.
76. Monaghan M, Helgeson V, Wiebe D. Type 1 diabetes in young adulthood. Curr Diabetes Rev. 2015; 11(4):239-250.
77. Mortensen H B, Hougaard P, Swift P, et al. New definition for the partial remission period in children and adolescents with type 1 diabetes. Diabetes Care. 2009; 32:1384-1390.
78. National Cancer Institute. Common Terminology Criteria for Adverse Events (CTCAE). https://ctep.cancer.gov/protocolDevelopment/electronic_applications/ctc.htm. Updated Mar. 1, 2018. Accessed Sep. 27, 2018.
79. Orban T, Bundy B, Becker D J, et al. Co-stimulation modulation with abatacept in patients with recent-onset type 1 diabetes: a randomised, double-blind, placebo-controlled trial. Lancet 2011; 378(9789):412-419.
80. Palmer J P, Fleming G A, Greenbaum C J, et al. C-peptide is the appropriate outcome measure for type 1 diabetes clinical trials to preserve beta-cell function: report of an ADA workshop, 21-22 Oct. 2001. Diabetes. 2004; 53(1):250-264.
81. Palmer J P. C-peptide in the natural history of type 1 diabetes. Diabetes Metab Res Rev. 2009; 25(4):325-328.
82. Rawshani A, Rawshani A, Franzén S, et al. Mortality and Cardiovascular Disease in Type 1 and Type 2 Diabetes. N Engl J Med. 2017; 376(15):1407-1418.
83. Rawshani A, Sattar N, Franzén S, et al. Excess mortality and cardiovascular disease in young adults with type 1 diabetes in relation to age at onset: a nationwide, register-based cohort study. Lancet. 2018; 392(10146):477-486.
84. Rigby M R, DiMeglio L A, Rendell M S, et al. Targeting of memory alefacept in new-onset type 1 diabetes (T1DAL study): 12-month results of a randomised, double-blind, placebo-controlled phase 2 trial. Lancet Diabetes Endocrinol. 2013; 1(4):284-294.
85. Rigby M R, Ehlers M R. Targeted Immune Interventions for Type 1 Diabetes: Not as Easy as it Looks! Curr Opin Endocrinol Diabetes Obes. 2014; 21(4):271-278.
86 Rigby M R, Harris K M, Pinckney A, et al. Alefacept provides sustained clinical and immunological effects in new-onset type 1 diabetes patients. J Clin Invest. 2015; 125(8):3285-3296.
87. Roark C L, Anderson K M, Simon L J, et al. Multiple HLA epitopes contribute to type 1 diabetes susceptibility. Diabetes. 2014; 63(1):323-331.
88. Rosenzweig M, Rosenthal C A, Torres V M, Vaickus L. Development of a quantitative assay to measure EBV viral load in patients with autoimmune type 1 diabetes and healthy subjects. J Virol Methods. 2010; 164(1-2):111-115.
89. Seaquist E R, Anderson J, Childs B, et al. Hypoglycemia and diabetes: a report of a workgroup of the American Diabetes Association and the Endocrine Society. Diabetes Care. 2013; 36(5):1384-1395.
90. Secrest A M, Becker D J, Kelsey S F, LaPorte R E, and Orchard T J. Characterising sudden death and dead-in-bed syndrome in Type 1 diabetes: analysis from 2 childhood-onset Type 1 diabetes registries. Diabet Med. 2011; 28(3):293-300.
91. Sherry N, Hagopian W, Ludvigsson J, et al., Protégé Trial Investigators. Teplizumab for treatment of type 1 diabetes (Protégé study): 1-year results from a randomised, placebo-controlled trial. Lancet. 2011; 378(9790):487-97.
92. Sorensen J S, Johannesen J, Pociot F, et al. Residual β-cell function 3-6 years after onset of type 1 diabetes reduces risk of severe hypoglycemia in children and adolescents. Diabetes Care. 2013; 36:3454-3459.
93. Sosenko J M, Skyler J S, Herold K C, et al. The metabolic progression to type 1 diabetes as indicated by serial oral glucose tolerance testing in the Diabetes Prevention Trial-type 1. Diabetes. 2012; 61(6):1331-7.
94. Steck A K, Dong F, Taki I, et al. Early hyperglycemia detected by continuous glucose monitoring in children at risk for type 1 diabetes. Diabetes Care. 2014; 37:2031-2033.
95. Steffes M W, Sibley S, Jackson M, Thomas W. Beta-cell function and the development of diabetes-related complications in diabetes control and complications trial. Diabetes Care. 2003; 26:832-836.
96 Streisand R. Young children with type 1 diabetes: challenges, resea directions. Curr Diab Rep. 2014; 14(9):520. pp 1-16.
97. Trancone A, Bonfanti R, Iafusco D, et al. Evaluating the experience of children with type 1 diabetes and their parents taking part in an artificial pancreas clinical trial over multiple days in a diabetes camp setting. Diabetes Care. 2016; 39:2158-2164.
98. Varni J W, Delamater A M, Hood K K, et al. PedsQL 3.2 Diabetes Module for children, adolescents, and young adults: reliability and validity in type 1 diabetes. Diabetes Care. 2018; dc172707.
99. Verkruyse L A, Storch G A, Devine S M, et al. Once daily ganciclovir as initial pre-emptive therapy delayed until threshold CMV load > or =10000 copies/ml: a safe and effective strategy for allogeneic stem cell transplant patients. Bone Marrow Transplant. 2006; 37(1):51-6.
100. Waldron-Lynch F, Henegariu O, Deng S, et al. Teplizumab induces human gut-tropic regulatory cells in humanized mice and patients. Sci Transl Med. 2012; 4(118):118ra12.
101. Wherrett D K, Chiang J L, Delamater A M, et al. Defining pathways for development of disease-modifying therapies in children with type 1 diabetes: a consensus report. Diabetes Care. 2015; 38(10):1975-85.
102. Ziegler A G and Nepom G T. Prediction and pathogenesis in type 1 diabetes. Immunity. 2010; 32:468-478.

1. A method of reducing exogenous insulin use in a subject in need thereof, the method comprising administering to the subject a 12-day to 14-day course of teplizumab at a total dose of from about 9000 μg/m² and about 14000 μg/m², wherein administration of teplizumab results in a reduction of exogenous insulin use by at least 0.08 U/kg/day.

2. The method of claim 1, wherein administration of teplizumab results in a reduction of exogenous insulin use by at least 0.08 U/kg/day at year 1.

3. The method of claim 1, wherein administration of teplizumab results in a reduction of exogenous insulin use by at least 0.10 U/kg/day at year 2.

4. The method of claim 1, comprising administering to the subject a 12-day course of teplizumab.

5. The method of claim 4, wherein the 12-day course of teplizumab comprises:
a first dose of about 106 μg/m² teplizumab on day 1,
a second dose of about 425 μg/m² teplizumab on day 2, and
one dose of about 850 μg/m² teplizumab on each of days 3-12.

6. The method of claim 1, comprising administering to the subject a 14-day course of teplizumab.

7. The method of claim 6, wherein the 14-day course of teplizumab comprises:
a first dose of about 100 μg/m² teplizumab on day 1,
a second dose of about 425 μg/m² teplizumab on day 2,
a third dose of about 850 μg/m² teplizumab on day 3,
a four dose of about 850 μg/m² teplizumab on day 4, and
a dose of about 1000 μg/m² teplizumab on each of days 5-14.

8. The method of any one of claims 1-7, comprising administering a first and a second 12-day to 14-day courses of teplizumab.

9. The method of any one of claims 1-7, comprising administering a first and a second 12-day courses of teplizumab.

10. The method of claim 8 or 9, wherein the two courses of teplizumab are administered at an interval of about 6 months.

11. The method of any one of claims 1-10, wherein the subject in need thereof has stage 3 type 1 diabetes.

12. The method of any one of claims 1-11, comprising administering teplizumab by intravenous infusion.

13. The method of any one of claims 1-12, wherein the subject in need thereof is about 7.5 years old or older.

14. The method of any one of claims 1-13, wherein reduction of exogenous insulin use is over a period of one year or more.

15. Teplizumab for use in reducing exogenous insulin use in a subject in need thereof in a method of any one of claims 1-14.

16. Use of teplizumab for the manufacture of a medicament for reducing exogenous insulin use in a subject in need thereof in a method of any one of claims 1-14.


SEQUENCE LISTING (2 sequences)

SEQ ID NO: 1 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK